SEQUENCE LISTING (43 sequences)

SEQ ID NO: 1 - DNA, 1542 bp, Yarrowia lipolytica
atgattatcatcgaaacgctcattggagcggtcgtttttgtggccgtgtacgtggcgtttgtcaagctcgactactaccgacgaaaagccaagtttgagacctcagacatgccggtcgcgtacaatggcttactgggctggaagggtctccggcacatgttgaccgtgttcaacaacgacattgggccggttgggtggcgggaggtgttcgccacgtacggaaagaccctcaagtactacgctttcccctccaacaccattttgacctacgaccccgataacatcaaagccatgctggcgacccagttcaaggacttttcgctgggtcttcgaaaggaggccctggctccgtcgctgggctacggaatcttcactcttgacgggtcgtcgtggtcgcattcccgcgctcttttgcggccccagttttcgcgagagcagatttctcggctcgaatctgtcgaaactcatgtgcaggaaatgatgagttgcattgacagaaaccagggtgcctatttcgacattcagcggctcttcttctccctggctatggacacggcgacagatttcctcctgggggaagctgtgggcaatctgcaagaaatcctgcatccggaaatgccccgaacaggaaccaccttccaggtggcgtttgaccgagcacagagactcgggtcgctgcgaatcatctgtcaggaagccttttgggtcgtgggaagcctgttctggagaagagacttcaataataccaaccagcatatccacgactatgttgatcggtacgtcgacaaggctcttctcgctcgaaaagaaaagtccgaaatctataccaatcccgacaagtacatctttttgtatgagctggctcgagaaaccacaaacaagatcactttgcgggaccaggtgctgaacattctgattgctggacgagatactacggcatccactttgtcgtggatcttcatggagcttgccaagaagccggatatcttccacaagctgagagaagcaattttgaacgactttggcacttcctgtgagtccatctctttcgagagtctcaagaagtgcgattatttgcgccaggtgctcaatgaaggtctcagattgcaccctgtggttcctgtcaatctgcgggtcgcggttagagacaccactcttcctcgaggaggaggtcctcaaggagacaagcccatctttgttgccaagggtcagaagatcaactacgccattttctggacccacagagacaaggagtactggggagaagatgctgaggagttccggcctgaaagatgggaaaccacatccgggggagctctaggaaagggctgggagtttctgccgttcaatggaggaccccggatctgtcttggtcagcagtttgccctcacagagatgggatatgtcatcactagactgcttcaggagtatagtgacattagtatccagccgtcggatgctgctgtcaaggtcagacattctctgaccatgtgtagcgcccagggtattaacatctcgctgaccagagccaaggaagagtga

SEQ ID NO: 2 - DNA, 1536 bp, Yarrowia lipolytica
atgttggaagtatttgctgccgccattgtcttcattgccgtatggcagacgtttctctggtacaatctcagagccagacaacgtaaatcaggctacggataccctccagtagcttctaatgggctgtttggctggagaggtctcaagtaccaactgacggtgttttctaaagatatcggtcccgagggatggggagaccaaatccaccggtacggaaagacccatctttatcctgtcgctcctacacagcttttggtcactacagatccggccaacatcaaggccattctcgctacccaattcaaggatttctccctgggacttcgatgcgaggctctttgcccttctcttggacacgggattttcactctggacggctccagttggtcgcactcacgagctctactaagaccccagttttcaagagagcagatctcccgtctagactctgtcgaagtccactttcaggaattggtaaagtgtattgacatgcactctggggagtattttgacatccagaaactcttctttgcccttgctatggacacttctaccgagtttctgctgggtgagtctgttggctgtctccaggagcttctgactccagatatgccccacaatggaaataacttccagtcagctttcgacagggttcagagacttggagctctgcgagttatcttccaagaaaactactggatcatcggcagtgtctttttccgtaaagagttccaggataccaatcggaccatccagagctttgtacagagatatgtggataaagcacttttggcccgaaaggaaaaggcacccatctacaccaacccagagaaatacgtctttttgtacgagttggctagagagaccaccaatggtaccgttttgagagatcaggtgctgaacattctcattgctggacgagacactacagcctctactctctcgtggatactgtttgagttggctcgtcatcctgagatttatcaaaagctccgaaaaacggttctggactgttttggtacttttaacgaagccatgagctttgaaagcctgaaacagtgcgacttccttcgacaagttctcaatgaaggtctcagactgtatcctgtggtgccaatcaacctgagagtagctctgaaagacactacactacccagaggtggtggacccaatggagatcagcccatattcgtgccaaaggatcagaaaatcaactatgccgtctactggacccatcgagacgtggattattggggagaagatgctgagactttccggccagaaagatggggaaacagcagtgctggacctcttggcaaaaactgggagtttctacccttcaatggcggtcccagaatttgtctcggacaacaatttgctctaacagagatgggctacattcttgctcgactcgtgcaagagtttagtttcatcagtctacagcaagacagggtgcctctaaaagttcgtcattccatcacaatgtgtcatggagatggcgtggttataaatctgactcgagctagggagctgtga

SEQ ID NO: 3 - DNA, 1545 bp, Yarrowia lipolytica
atgatactcatcgaaacattcattggagcagcggtctttctggccgtataccttgggtttgtgcaactcgacttgtaccgaagaaaacgcaagtttgagacctacgactttccagtggcacgtaacggattttggggctggaagggactacgacattttctgcacgtcttcaacaatgatctcggacctatgggatggagaggtgttttcgctcagtatggaaagactctcaagttttatgccttccccagtaacaccattctgacctatgatcctgacaacatcaaagccatgctggccactcaattcaaagacttttcactaggtcttcgaaaagaggcgcttgccccttctctgggctacggaatcttcactcttgacggctcctcctggtctcattctcgagctctgttgcgtccgcaattttcccgagaacagatttctcgtcttgaatctgtcgagaatcatgtccaggagatgatgagcatcattgacaagcagcagggcaagtctttcgacattcagcgactcttcttctgtctggctatggatacagctactgatttcctgctaggagaatctgtgggtaatctgcaagagattctgcatcctgaaatgccccgagcaggtgccagtttccaggtggcgtttgatcgtgcccagagactgggatctttgcgaattatctgccaggaggccttttgggtcgttggaagtgtgttttgccgacgagaattcaacagaactaacaaacatatccacgactttgttgaccaatacgtggataaggcgcttttggctcgaaaggagaaatctgaaatctacaccaatcccgacaaatacatctttctgtacgagctggctcgagaaactacaaacaagattactctacgagatcaggtgttgaacattctgattgctggacgagataccactgcctctactctctcatggatcttcatggagcttgccaagaagcccgagatctaccacaagttgagagaagctgttttggacgactttggcacctcttgtgattccatctcttttgagagtctcaagaagtgtgagtacttgcgacaggtgctcaatgaaggtctcagattgcaccctgtggtccccattaacctgcgagttgccgttagagacaccactcttcctcgtggaggtggtcctcaaggagacaagcccattttcgttgccaagggtcagaagatcaactacgccatctactggactcatcgagacaaggagtactggggagaagacgctgaggagttccgacccgaacgatggggcgaaactgctgctggaggagtccttggaaagggctgggagttcttgccgttcaatggaggtccccgaatctgtcttggacagcagtttgctcttactgagatgggatacgtgattgtgcgactgttgcaagagtacagcgatatcagtgcggagccgtctgtggctgatgtcaaggtccgtcattcgctgacaatgtgcagtgccgacggtatcaacatttcgttggcgagggcaaacaatgagtag

SEQ ID NO: 4 - DNA, 1542 bp, Yarrowia lipolytica
atgctaattctcgaatctctggtcggagtggccctgtttgtggctctgtacatcgggttcacgcaactcgatttatacagacgaaaacgcaagtttgaaaactacgactttcccgtcgcatacaatggattccttggatggaagggtctacgtcacatgttgacggttttcaacaacgacattggtcctctaggttggagacccgtgtttgaggccaatggcaagactttcaagttctacgccttcccttcaaacagtattctcacttatgatcccgacaacatcaaggccatgctggcgtctcaattcaaggacttttctctgggtttgcgaaaggaggctctggcgccgtctctaggacacggaatcttcactctggatggctcgtcgtggtcccattctcgagctctactacgtccccagttttccagagagcagatttcgcgtctggaatctgtggaaacccacattcaggaaatgatgagcatcgttgacaagaaccagggcgagtactttgacatccagcgactcttcttctccctggcaatggataccgctacagactttctgctaggagaagccgtgggcaacctgcaggagattctgcatccggaaatgccccgtaccggaaccgccttccaggtggcgtttgatcgtgcccagcgaatggggtctctgcgaatcattcttcaagacgctttctgggtggtcggaagcctgtttaagaaggcagagtttgaaagaacaaacaagcacatccatgactatgtcgatcgttacgtcgataaggcactgctggcgcgacaggaaaagtctgaaatctatactaaccccgacaagtacatctttttgtacgagctggctcgtgaaactacaaacaaaaaggtgctgcgtgaccaggtgctcaacattctgattgccggacgagataccacggcgtcctcgctgtcgtggatctttatggagctcgccaggaaacccgagctgttcccaaagctcagacaggccattttgaacgactttggaacctcctgcgagtccattagctttgagtcgctcaaaaagtgcgattatctgcgctacgtgctcaatgagggactgcgtcttcatcccgtggttcctgtcaatctccgagttgctcttcgagacacaactcttcctcgaggaggaggccccaacggagacaagcctatcttcctggccaagggccagaaggtcaactactccattttctggacccatcgagccaaggagtactggggcgaagacgctgaggagttccgacctgaacggtgggatacaacctcagaaggccctctaggacgaggatgggagtttttgcccttcaatggaggtccccgaatctgtcttgggcagcagtttgcacttaccgaaatgggatatgtcattacccggctgcttcaggagtatagtgatatcagtatccggccttctgactctcccatcaaggtcagacattctctgacaatgtgcagtgccgagggtattcatatttcgttgaccagagccaagagcgggtga

SEQ ID NO: 5 - DNA, 1056 bp, Yarrowia lipolytica
atgtctgctcccgtcatccccaagacccagaagggtgtcatcttcgagacctccggcggtcctctcatgtacaaggacatccccgtgcctgtgcctgccgacgacgagattctggtcaacgtcaagttctccggagtctgccacacggatctgcacgcctggaagggcgactggcctctggacaccaagcttcctctggtcggaggccacgagggtgccggagtggttgttgccaagggtaagaacgttgacacgtttgagattggcgactatgccggcatcaagtggatcaacaaggcctgctacacctgcgagttctgccaggtggccgccgagcccaactgtcccaacgctaccatgtctggatacacccacgacggctctttccagcagtacgccaccgccaacgccgtgcaggccgcgcacattcccaagaactgcgatctcgccgagattgcccccattctgtgcgccggaatcaccgtctacaaggctctcaagactgccgccatcctcgctggccagtgggttgccgttactggtgctggaggaggactcggaacacttgctgtccagtacgccaaggccatgggctaccgagtgctggccattgacactggcgccgacaaggagaagatgtgcaaggaccttggtgccgaggttttcatcgactttgccaagaccaaggacctcgtcaaggacgtccaggaggccaccaagggcggaccccacgccgtcatcaatgtgtctgtctccgagtttgcagtcaaccagtccattgagtacgtgcgaaccctgggaaccgttgttttggtcggtctgcccgccggcgccgtctgcaagtctcccatcttccagcaggtggctcgatctatccagatcaagggctcttacgttggaaaccgagccgactcccaggaggccattgagttcttctcccgaggtctcgtcaagtcgcccatcatcatcatcggtctgtccgagctggaaaaggtctacaagcttatggaggagggcaagattgccggccgatacgttctggacacctccaagtaa

SEQ ID NO: 6 - DNA, 1276 bp, Yarrowia lipolytica
agtgagtttgcccacataggtacagtcccagactactcctgaaacacagaggtagatgcccagaccttcactttgaccgctacacaaacaattgcgcagagtgtcgtgccctgtatcacagtctgcacagtgccctgcttgtgtgcatcgcattgcattgattctgtagctctccgcagggagtatttccccatataccaatgctaacacagtgagcgacgttcccaagacacaaaaggccgtcgttttcgaggaagtcaacggacctttgatgtacaaggacattcccgtccccactcccgccaaggacgagctgctcgtcaaggtgcagtattccggtgtctgccactcggatctgtccatctggaagggtgattgggcacagcagctgcggttcagccccaagatgccgctggtcggcggtcatgagggagcaggagaggttgtgggcatgggcgatcaggtgaccggatggcaggtcggagaccgaaccggagtcaagtttatttctggctcttgtctcacttgcgagcactgttctgctggctgggaccagcactgcgtagcccccggcgtgtcaggtctgctcaaagacggctctttccagcagtacgcctgcgtgaaggccgccaccgcaccccgaatccccgattcttgcgatctggctggtgttgcacccgttctgtgtgcaggcatcaccgcctacactgccctcaagaactctggtctcaaggccggtgagtgggtggtgatcaccggagctggaggaggactcggatcctacgccgtccagtacgccaagtgcatgggtttccgtgtgattgccattgacactggagacgacaaggagacccacaccaaggagctgggagccgaggtgtttattgactttgccaagagtggtgctggcatgattgctgagattcacaagctcaccggaggtggcgcccacgccgtggtcaactttgctgtgcaggacgcggctgtcgaggctgccactctgtacgtgcgaacccgaggcactctggttctgtgtgctctgccacccaacggtaccgtcaagagtcacattctcaaccacgtgggtcgaggactcaccatcaagggcagttatgtgggtaataagctggatactcaggaagccattgacttctatgcacggggtctcgtcaagaccaagtaccgtctcggcgagctgagcaagctcgaggagtattaccagcagatgcttgatggtaagattgttggtcgtgtcgttgttgataacagcaagtag

SEQ ID NO: 7 - DNA, 1590 bp, Yarrowia lipolytica
atgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactag

SEQ ID NO: 8 - DNA, 1590 bp, Yarrowia lipolytica; identical to SEQ ID NO: 7

SEQ ID NO: 9 - DNA, 1830 bp, Yarrowia lipolytica
atgtctgacgacaagcacactttcgactttatcattgtcggtggaggaaccgccggccccactctcgcccggcgactggccgatgcctggatctccggtaagaagctcaaggtgctcctgctcgagtccggcccctcttccgagggtgttgatgatattcgatgccccggtaactgggtcaacaccatccactccgagtacgactggtcctacgaggtcgacgagccttacctgtctactgatggcgaggagcgacgactctgtggtatcccccgaggccattgtctgggtggatcctcttgtctgaacacctctttcgtcatccgaggaacccgaggtgatttcgaccgaatcgaagaggagaccggcgctaagggctggggttgggatgatctgttcccctacttccgaaagcacgagtgttacgtgccccagggatctgcccacgagcccaagctcattgacttcgacacctacgactacaagaagttccacggtgactctggtcctatcaaggtccagccttacgactacgcgcccatctccaagaagttctctgagtctctggcttctttcggctacccttataaccccgagatcttcgtcaacggaggagccccccagggttggggtcacgttgttcgttccacctccaacggtgttcgatccaccggctacgacgctcttgtccacgcccccaagaacctcgacattgtgactggccacgctgtcaccaagattctctttgagaagatcggtggcaagcagaccgccgttggtgtcgagacctacaaccgagctgccgaggaggctggccctacctacaaggcccgatacgaggtggttgtgtgctgcggctcttatgcctctccccagcttctgatggtttccggtgttggacccaagaaggagctcgaggaggttggtgtcaaggacatcattttggactctccttacgttggaaagaacctgcaggaccatcttatctgcggtatctttgtcgaaattaaggagcccggatacacccgagaccaccagttcttcgacgacgagggactcgacaagtccaccgaggagtggaagaccaagcgaaccggtttcttctccaatcctccccagggcattttctcttacggccgaatcgacaacctgctcaaggatgatcccgtctggaaggaggcctgcgagaagcagaaggctctcaaccctcgacgagaccccatgggtaacgatccctctcagccccatttcgagatctggaatgctgagctctacatcgagctagagatgacccaggctcccgacgagggccagtccgtcatgaccgtcatcggtgagattcttcctcctcgatccaagggttacgtcaagctgctgtcccccgaccctatggagaaccccgagattgtccacaactacctgcaggaccctgttgacgctcgagtcttcgctgccatcatgaagcacgccgccgacgttgccaccaacggtgctggcaccaaggacctcgtcaaggctcgatggcccccggagtccaagcccttcgaggaaatgtccatcgaggaatgggagacttacgtccgagacaagtctcacacctgtttccacccctgtggtactgtcaagcttggtggtgctaatgataaggaggccgttgttgacgagcgactccgagtcaagggtgtcgacggcctgcgagttgccgacgtctctgtccttccccgagtccccaacggacacacccaggcttttgcctacgctgttggtgagaaggctgccgacctcatccttgccgacattgctggaaaggatctccgacctcgaatctaa

SEQ ID NO: 10 - DNA, 1830 bp, Yarrowia lipolytica
atgtctgacgacaagcacacttacgacttcattattgtcggtggaggaaccgctggccccactctcgcccgacgactggctgatgcctggatctcgggcaagaagctgaaggtgcttctgctcgagtcgggcccttcttctgagggtgtcgaggatattcgatgccccggaaactgggtcaacaccatccactccgattacgactggtcctacgaggccgacgagccctacctgtctactgacggtgaggagcgacgaatctgcggtatcccccgaggccactgccttggaggatcctcttgtcttaacacctcttttgtcatccgaggaacccgaggtgactttgaccgaatcgaggaggagaccggcgccaagggctggggatgggacgatctgttcccctacttccgaaagcacgagactttcgtgccccagggatctgctcacgagcccaagctcatcgactttgacgcctacgactacaagaagttccacggtgactctggtcccatcaaggtccagccttacgactacgcccccatctccaagaagttctccgagtctctcgcttctttcggatacccttacaaccccgagatctttgtcaacggaggagccccccagggttggggtcacgttgtccgatccacctccaacggtgttcgatccactggctacgacgctctcgtccacgcccccaagaacctcgacattatcaccggccacgttgccaccaagattctctttgagaagattggaggcaagcagaccgccgtcggtgtcgagacctacaaccgagctgccgaggaggctggccctaccttcaaggcccgatacgaggttgttctgtcctgcggctcctacgcctctccccagcttctgatggtttccggtgtcggacccaagaaggagcttgaggctgttggtgtcaaggacatcatcctggactctccctacgtcggaaagaacctgcaggaccatcttatctgcggtatctttgtcgagatcaaggagcccggatacaccagagaccaccagttcttcgacgatgagggactcgaaaagtccaccgaggagtggaagagcaagcgaaccggtttcttctccaaccctccccagggtatcttctcttacggccgagtcgacaacctgctcaaggacgatcccgtgtggaccgctgccagcgagaagcagaaggctatcaacccccgacgagaccccatgggcaacgacccctctcagccccattacgagatatggaacgccgagctgtacattgagctggagatgacccaggctcccgacgagggcgagtctgtcatgaccgttatcggtgagattctgccccctcgatccaagggttacatcaagctgttgtctgctgaccctctcgagaagcccgagattgtccacaactacctgcaggaccctgtcgacgctcgagtgtttgctgctatcatgaagcacgccgccgacgttgccaccaacggtgctggcaccaaggacctcgtcaaggctcgatggcccccggagtccacccccttctccgagatgtccatcgaggactgggagaactacgtccgagacaagtcccacacctgtttccacccctgtggtaccgtcaagcttggtggtgctaacgataaggaggccgttgtcgacgagcgactccgagtcaagggtgttgacggcctgcgagttgccgatgtctctgtcctcccccgagtccccaacggacacacccaggcctttgcctacgctgtcggtgagaaggctgccgatctcatccttgccgacattgcttgccgggatctccgacctcgaatctag

SEQ ID NO: 11 - DNA, 1830 bp, Yarrowia lipolytica
atgtctgacgacaagcacacttacgactttatcattgtcggtggaggaaccgccggccccactcttgcccgacgactggctgaggcctggatctccggcaagaagctcaaggtccttctcctggagtccggtcctacctctgagggtgttgaggatattcgatgccccggaaactgggtcaacaacatccactccgagcacgactggtcttacgaggtcgatgagccctacctttctaccgatggagaggagcgacgaatctgcggtatccctcgaggccactgtctgggaggatcctcttgtcttaacacctctttcgtcatccgaggaactcgaggagactttgaccgaatcgaggaggagactggcgccaagggctggggatgggacgatctgttcccttacttccgaaagcacgagtgctacgttccccagggacccgcccacgagcccaagctcatcgactttgagagctacgacttcaagaagttccacggtgactctggccccatcaaggtccagccttacgactacgcccccatctccaagaagttctccgagtccctggcttctttcggatacccttacaaccccgagatcttcgtcaacggaggagctccccagggctggggccacgtgatccgatccacttccgatggtatccgatccaccggctacgacgctctcgtccacgcccccaagaacctcgacgttgtcactggccacgccgtcaccaagattctgtttgaggagattggaggcaagcagaccgccgtcggagtcgagacctacaaccgaggcgccgaggaggctggccctacctacaaggctcgatacgaggttgtcatctgctgcggctcttacgcttctccccagcttctgatggtttccggtgtcggaccccgaaaggagctcgaggaggttggtgtcaaggacatcattctggactctccctacgtcggaaagaacctgcaggaccatcttatctgcggtatctttgtcgagatcaaggagcccggatacacccgagaccaccagttcttcgatgacgagggactcgagaagtctaccgaggagtggaagtccaagcgaactggcttcttctccaaccctccccagggtatcttctcttacggccgaatcgacaacctgctcaaggacgatcccgtgtggaaggctgcctgcgagaagcagaaggccatcaacccccgacgagaccccatgggcaacgatccttctcagccccatttcgagatctggaacgccgagctgtacattgagctggagatgacccaggctcccgatgagggcgagtccgtcatgaccgtcattggtgagattctgcctcctcgatccaagggatacgtcaagctgctgtcccccaaccctctggagaaccccgagattgtccacaactacctgcaggaccctctcgatgcccgagtcttcgccgccatcatgaagcacgctgctgacgttgccaccaacggtgctggcaccaaggacctcgtcaaggcccgatggcccccggagtccaagcccttcgaggacatgtccatcgaggagtgggagacctacgtccgagacaagtctcacacctgtttccacccctgtggtaccgtcaagcttggtggtgctaacgataaggaggctgttgttgacgagcgtctccgagtcaagggagttgacggtctgcgagttgccgacgtgtctgttcttccccgagtccccaacggacacacccaggcctttgcctacgctgttggagagaaggctgccgatctcattcttgccgacattctcggccgagatctccgacctcgaatctaa

SEQ ID NO: 12 - DNA, 1830 bp, Yarrowia lipolytica
atggctgacgacaaacacactttcgactttatcattgtcggtggcggcaccgccggccccaccctggcccgacgtctcgccgaggccaacatctccggcaagaagctaaaggtcctgcttctcgagtccggtccctcttccgagggcgtggaggatatccgatgtcccggaaactgggtcaacaccatccactccgactacgactggtcctacgaggtcgacgagccctacctctccaccgagggcgaaaagtcccgacagtgcggaatcccccgaggccactgtctcggcggctcgtcgtgcctcaacacctcgttcatcatccgaggaacccgaggcgactttgaccgaatcgaggaggagaccggcgccaagggctggggctgggacgacctgttcccctacttcctcaagcacgagtgcttcgtgccccagggacccgcccacgagcccaagcgaatcgactttgacacctacgactacaagaagttccacggagactccggccccatcaagtgccagccctacgactacgcgcccatctccaagaagttctccgagtcgctgacctcgtacggatacccctacaaccccgagatcttcgtcaacggaggagctcccgagggctggggccatgtggtccgatccaccgccaacggtgtgcgatccaccggatacgacgccctggtgcacgcccccaagaacctgaccgttctcaccggccacgccgtcaccaaggtgctgttcgagaagattggcggcaagcagaccgccgtcggcgtccagacccacaaccgagagaccgccgaggctggccccgtcttcaaggcccgatacgaggtcattctctcttgcggctcctacgcctccccccagctcctcatggtgtctggtgtcggcccccagaaggagctcgaggccgttggtgtcaaggacatcattctcgactctccctacgtcggcaagaacctgcaggaccatcttatctgcggtatcttcgtcgagatcaacgagcccggctacacccgagaccaccagttcttcgacgacgagggcctggagctgtcgaccgccgagtggaaggacaagcgaaccggattcttctccaaccctccccagggaatcttctcctacggccgaatcgagaagctgctgaaggacgatcccgtgtggatcgaggcctgcgagaagcagaagaagctcaaccccaagcgagaccccatgggcaacgaccccacccagccccatttcgagatctggaacgccgagctgtacattgagctcgagatgacccaggctcccgacgagggccagtccgtcatgaccgtcattggcgagatcctgccccctcgatccaagggatgggttaagctcaagtctgccgatcctctcgagaaccccgacatccagcacaactacctccaggaccccgtggacgcccgagtcttcgccgccatcatgaagcacgccgccgacatggccaccaacggtgctggcaccaaggaccttgtcaaggcccgatggcccccggagtccaagccctttgctgacatgtccatcgaggagtgggagacgtacgtgcgagacaagtcgcacacctgtttccacccctgtggtacctgtaagctcggtggagccaacgacaaggaggctgttgttgacgagcgactccgagtcaagggtgttgatggcctgcgagttgtcgacgtttccgtccttccccgagtccccaacggccacactcaggcttttgcctacgctgttggcgagaaggctgccgatctcattattcaggacattcttggtcgagatctccgaccccgaatttaa

SEQ ID NO: 13 - DNA, 1830 bp, Yarrowia lipolytica
atgtctgacgataagcacacttacgatttcattattgttggcggaggtactgccggcccaaccctggctcgaagactggctgaggcctggatctctggcaagaagctcaagatcctccttgtcgagtctggcccttcgtctgagggtgtggaggatattcggtgtcccggaaactggatcaacaccatcacctccgagtacgactggtcatacgaggttgatgaacctcatctcaccactgaaggtgaaaagtctcgtcactgtggtattcctcgtggccattgtcttggtggatcttcgtgtctgaacacttcttttgtcattcgaggaacccgaggagactttgaccgaattgaggaggaaactggcgccaaaggctggggttgggatgacttgttcccctacttcctgaagcacgagtgctatgttccccaaggaccagcccatgagcctaaactcattgatttcgacacatatgattacaagaagttccacggaagctctggtcccatcaaggtccaaccttacgattatgctcctatttcaaagaagttctctgagtctctgtcttccttcggatacccctacaaccctgagatctttgccaatggtggtgctcccatgggatggggtcatgttgttcgatccacatccaatggtgtcagatctacaggctatgatgctctcgtatacgcccccaagaacctggaagtcatcactggccacgccgtaaccaagattctttttgagaacattggtggcaagcccactgctgttggtgttcagacttataatcgagccgcagaagaggctggccctgtcttcaaagcgcgatatgaggtgatcctgtgttgtggttcttatgcctctcctcaactcctcatggtgtcaggtattggtcccaagaaggagcttgaagctgttggtgttaaggatattgttcttgactctccctatgtgggaaagaaccttcaggaccatctcatctgtggtatctttgttgagatcaaggagcctggctacactcgtgaccatcagttctttgatgatgacggccttgagaaatctaccgaggagtggaagacaaagcgaactggattcttctccaaccctcctcagggtatcttctcttatggtcgaattgacaatttgctgaaggatgatcccgtctggactgccgcttgtgagaaacagaagaagattaaccctcggcgagacccaatgggcaatgatccaacccagccccattttgagatctggaacgccgagctctatattgagcttgaaatgactgctgctcctgatgagggacaatctgtcatgactgtcattggagaaattttgcctcctcgatctacaggatacatcaagctcaagtctcctgatgttatggagaaccctgaaattgtccacaattacctacaagatcctgttgatgctcgagtcttcgcagctatcatgaagcatgctgctgacattgccaccaatggagctggtaccaaggatcttgttaaggccagatggcccccggagtccaagccttacgctgatatgtccattgaagagtgggagcagtacgtccgtgacaagtctcacacctgtttccacccctgtggtactgtaaagcttggtggtgccaacgacaaggaggctgttgttgacgagcgactccgagtcaagggtgttgacggtctacgagtggctgatgtttctgttctccctcgtgttcccaatggacacactcagtcttttgcctacgctgtgggtgagaaggctgcggatcttattcttgcggacatcctcggccgagatctccgacctcgaatttaa

SEQ ID NO: 14 - DNA, 2169 bp, Yarrowia lipolytica
atgcctctactcgactctctcgactttattgttctggtgctggtgggcgtggccaccctggcctttttcaccaagggcaagttgtgggccaaggagcccgagacggacccctatgcaggtggtctgggctcgcagggcttcggatccaccacctcgttcggatcgttcggaggcaactccaacaagacccgagacattaccaagaagctggagcagaccggcaagaacgtgatcatcttctacggctcgcagaccggaactgccgaggattacgccaaccgactgtccaaggaagcaacccagcgatatggcctcaagtccatgaccgccgatctcgaggactacgactacgagaacctcaactcactgggcgacgacattgtcgtgggttttgtcatggccacttacggcgagggagagcccaccgataacgctgtcaacttctacggattcatcaacgacggctcttccgagtgggccgaatccgacgagccctctgccgaccccgactctcccctgtcttctctcaactacgtcattttcggtcttggaaacaacacctacgaacactacaacgagattggccgaaacctggacaagcgactcaagaagctgggcgccaagcgaattggtgactacggcgagggtgacgatggacagggcaccatggaagaggactacctcgcctggaaggacgaccttttctccgcctggaaggaggccaagggtctggacgagcatgaggccaagtatgagccctccgtcaagatctccgagaccggcgagaccggctcttccgaggactcctcttctgttgctgagcctgatgctgaggccatgtctgtgtacctgggtgagcctaacaagaagattctccgaggcgagatcaagggcccctacaacgccggtaaccccttcttggctaacgtttccgagacccgagagctgttccacgaccccaagcgatcctgtatccacgtcgagtttgatgttggcaccaacgtcaagtacaccaccggtgaccatcttgctctgcacattcagaactccgacgaagaagttgagcgattcctcaaggtcattggtctctgggacaagcgacacaatgtcatcaaggccaagcccattgatcccgcctacaagccctctcttcctgtccctactacctatgatactgttgtccgatactacctggagatcaatggtgctgtttcccgacagctgctggccttcattgcccctttcgcccccaccgagactgctaagaaggaggctctgcgacttggttccgacaagaacgcttttgccgatgaggttgccaagcactacaccaacattgcccatgttctctccaagctgtctggcgacgagccttggaccaacgtgcccttctccttcctggttgagtctctcccccatctgatcccccgatactactccatctcgtcctcttccttggtggacaagtccaagatctccatcaccgccgtggtcgagtcccttgaggcccccgagtacgccatcaagggtgttgccaccaacctgctgcttgacatgaagatcaagaaggatggtgttgacccctcaaagtcaaaggacccccaagccgtgcactacgagctgagtggtccccgaggcaagttttggggccacaagctccccgtgcataccagacagtctaacttcaagctgccctctgaccccaagaagcccatcattatgattggtccaggaactggtcttgctcccttccgagcctttgtcatggagcgagctaagcaggccgaaagcggcaccgacgtgggtcaacagcttctcttctttggctgccgaaaccccaacgaggatttcatctacaaggagcagtgggccggcattgagaaggagctcggtgacaagttcaccatggtcactgctttctcccgagtcgaccccgtccaaaaggtctatgtccaacaccgaatgcaggaatatgccaagcagatcaacgatctcatgcaacagggcgcctacttttacgtgtgtggagacgcctcgcgaatggcccgagaggttcaggccaccctggccaagattctgtctgatcagcggggcattcccctgtcttctgctgagcagctggtcaagagcctcaaggtgcagaacgtctaccaggaagatgtgtggtaa

SEQ ID NO: 15 - DNA, 1545 bp, Yarrowia lipolytica
atgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaa

SEQ ID NO: 16 - DNA, 1581 bp, Yarrowia lipolytica
atggaagtccgacgacgaaaaatcgacgtgctcaaggcccagaaaaacggctacgaatcgggcccaccatctcgacaatcgtcgcagccctcctcaagagcatcgtccagaacccgcaacaaacactcctcgtccaccctgtcgctcagcggactgaccatgaaagtccagaagaaacctgcgggacccccggcgaactccaaaacgccattcctacacatcaagcccgtgcacacgtgctgctccacatcaatgctttcgcgcgattatgacggctccaaccccagcttcaagggcttcaaaaacatcggcatgatcattctcattgtgggaaatctacggctcgcattcgaaaactacctcaaatacggcatttccaacccgttcttcgaccccaaaattactccttccgagtggcagctctcaggcttgctcatagtcgtggcctacgcacatatcctcatggcctacgctattgagagcgctgccaagctgctgttcctctctagcaaacaccactacatggccgtggggcttctgcataccatgaacactttgtcgtccatctcgttgctgtcctacgtcgtctactactacctgcccaaccccgtggcaggcacaatagtcgagtttgtggccgttattctgtctctcaaactcgcctcatacgccctcactaactcggatctccgaaaagccgcaattcatgcccagaagctcgacaagacgcaagacgataacgaaaaggaatccacctcgtcttcctcttcttcagatgacgcagagactttggcagacattgacgtcattcctgcatactacgcacagctgccctacccccagaatgtgacgctgtcgaacctgctgtacttctggtttgctcccacactggtctaccagcccgtgtaccccaagacggagcgtattcgacccaagcacgtgatccgaaacctgtttgagctcgtctctctgtgcatgcttattcagtttctcatcttccagtacgcctaccccatcatgcagtcgtgtctggctctgttcttccagcccaagctcgattatgccaacatctccgagcgcctcatgaagttggcctccgtgtctatgatggtctggctcattggattctacgctttcttccagaacggtctcaatcttattgccgagctcacctgttttggaaacagaaccttctaccagcagtggtggaattcccgctccattggccagtactggactctatggaacaagccagtcaaccagtactttagacaccacgtctacgtgcctcttctcgctcggggcatgtcgcggttcaatgcgtcggtggtggttttctttttctccgccgtcatccatgaactgcttgtcggcatccccactcacaacatcatcggagccgccttcttcggcatgatgtcgcaggtgcctctgatcatggctactgagaaccttcagcatattaactcctctctgggccccttccttggcaactgtgcattctggttcacctttttcctgggacaacccacttgtgcattcctttattatctggcttacaactacaagcagaaccagtag

SEQ ID NO: 17 - DNA, 1947 bp, Yarrowia lipolytica
atgacacaacctgtgaatcggaaggcgactgtcgagcgggtcgagccagcagtggaggtggctgactccgagtccgaggccaagaccgacgtccacgttcaccaccatcatcaccaccacaagcgaaaatccgtcaagggcaagattctcaacttcttcacccgaagtcgacgtatcaccttcgtcctcggcgccgtggtcggtgtgatagccgcgggatactacgctgcgccaccggagctcagcattgatatcgatgctcttctcggcgacttgccctcgttcgactttgacgctctatctctcgacaacttgtccatggacagtgtgtcggactttgtacaagacatgaaatcgcggtttccgaccaagattctgcaggaggcggccaagatcgagaagcaccagaaaagcgaacagaaggctgccccttttgctgtgggcaaggctatgaagagcgagggactcaacgccaagtacccggtggtgctggtgcccggcgtcatctccacgggactggagagctggtccctggagggaaccgaggagtgtcccaccgagtcgcacttcagaaagcgaatgtggggctcctggtacatgatccgagtcatgctgctggacaagtactgctggctgcagaacctgatgctggacacagagaccggtctagaccctccccatttcaagctgcgagccgcccagggatttgcctccgccgacttctttatggcaggctactggctgtggaacaagctgctcgagaacctggctgttattggatacgatacggatacaatgtctgctgcggcgtacgactggagactgtcctaccctgatttggagcaccgagacggatacttctccaagctcaaagcttcaatcgaagagactaagcgtatgacaggtgagaagacagttctgacgggccattccatgggctcccaggtcatcttctacttcatgaagtgggctgaggccgagggatatggaggaggaggtcccaactgggtcaatgaccatattgaatcctttgtcgacatttccggctccatgctgggtactcccaagaccctggttgctcttctgtctggagaaatgaaggataccgtgcagctgaacgcgatggctgtgtatggactggagcagttcttctctcgacgagagcgagccgatctgctgcgaacatggggaggaattgcttccatgattcccaagggtggtaaggctatctggggtgatcattctggagcccctgatgacgagcctggccagaatgtcacctttggcaacttcatcaagttcaaggagtccttgaccgagtactctgctaagaacctcaccatggatgaaaccgttgacttcctgtattctcagtctcccgagtggtttgtgaaccgaaccgagggtgcttactcctttggaattgccaagactcgaaagcaggttgagcagaatgagaagcgaccttctacctggagcaaccctctggaagctgctctccccaatgcccccgatctcaagatctactgcttctatggagtcggtaaggataccgagcgagcctactactaccaggatgagcccaatcccgagcagaccaacttgaacgtcagtatcgctggaaacgaccctgatggtgtgcttatgggccagggcgatggaaccgtctcccttgtgacccataccatgtgtcaccgatggaaggacgagaattccaagttcaaccctggtaacgcccaggtcaaggttgtggagatgttgcaccagcctgatcgacttgatattcgaggcggtgctcagactgccgagcatgtggacattctggggcgttctgagttgaacgagatggttctgaaggtggccagtggaaagggaaatgagattgaagagagagtcatctccaacattgatgagtgggtgtggaagattgatctcggcagcaattag

SEQ ID NO: 18 - DNA, 7549 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatcccacaatgattatcatcgaaacgctcattggagcggtcgtttttgtggccgtgtacgtggcgtttgtcaagctcgactactaccgacgaaaagccaagtttgagacctcagacatgccggtcgcgtacaatggcttactgggctggaagggtctccggcacatgttgaccgtgttcaacaacgacattgggccggttgggtggcgggaggtgttcgccacgtacggaaagaccctcaagtactacgctttcccctccaacaccattttgacctacgaccccgataacatcaaagccatgctggcgacccagttcaaggacttttcgctgggtcttcgaaaggaggccctggctccgtcgctgggctacggaatcttcactcttgacgggtcgtcgtggtcgcattcccgcgctcttttgcggccccagttttcgcgagagcagatttctcggctcgaatctgtcgaaactcatgtgcaggaaatgatgagttgcattgacagaaaccagggtgcctatttcgacattcagcggctcttcttctccctggctatggacacggcgacagatttcctcctgggggaagctgtgggcaatctgcaagaaatcctgcatccggaaatgccccgaacaggaaccaccttccaggtggcgtttgaccgagcacagagactcgggtcgctgcgaatcatctgtcaggaagccttttgggtcgtgggaagcctgttctggagaagagacttcaataataccaaccagcatatccacgactatgttgatcggtacgtcgacaaggctcttctcgctcgaaaagaaaagtccgaaatctataccaatcccgacaagtacatctttttgtatgagctggctcgagaaaccacaaacaagatcactttgcgggaccaggtgctgaacattctgattgctggacgagatactacggcatccactttgtcgtggatcttcatggagcttgccaagaagccggatatcttccacaagctgagagaagcaattttgaacgactttggcacttcctgtgagtccatctctttcgagagtctcaagaagtgcgattatttgcgccaggtgctcaatgaaggtctcagattgcaccctgtggttcctgtcaatctgcgggtcgcggttagagacaccactcttcctcgaggaggaggtcctcaaggagacaagcccatctttgttgccaagggtcagaagatcaactacgccattttctggacccacagagacaaggagtactggggagaagatgctgaggagttccggcctgaaagatgggaaaccacatccgggggagctctaggaaagggctgggagtttctgccgttcaatggaggaccccggatctgtcttggtcagcagtttgccctcacagagatgggatatgtcatcactagactgcttcaggagtatagtgacattagtatccagccgtcggatgctgctgtcaaggtcagacattctctgaccatgtgtagcgcccagggtattaacatctcgctgaccagagccaaggaagagtgacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 19 - DNA, 7549 bp, Artificial sequence; identical to SEQ ID NO: 18
  vector

SEQ ID NO: 20 - DNA, 7062 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatccacaatgtctgctcccgtcatccccaagacccagaagggtgtcatcttcgagacctccggcggtcctctcatgtacaaggacatccccgtgcctgtgcctgccgacgacgagattctggtcaacgtcaagttctccggagtctgccacacggatctgcacgcctggaagggcgactggcctctggacaccaagcttcctctggtcggaggccacgagggtgccggagtggttgttgccaagggtaagaacgttgacacgtttgagattggcgactatgccggcatcaagtggatcaacaaggcctgctacacctgcgagttctgccaggtggccgccgagcccaactgtcccaacgctaccatgtctggatacacccacgacggctctttccagcagtacgccaccgccaacgccgtgcaggccgcgcacattcccaagaactgcgatctcgccgagattgcccccattctgtgcgccggaatcaccgtctacaaggctctcaagactgccgccatcctcgctggccagtgggttgccgttactggtgctggaggaggactcggaacacttgctgtccagtacgccaaggccatgggctaccgagtgctggccattgacactggcgccgacaaggagaagatgtgcaaggaccttggtgccgaggttttcatcgactttgccaagaccaaggacctcgtcaaggacgtccaggaggccaccaagggcggaccccacgccgtcatcaatgtgtctgtctccgagtttgcagtcaaccagtccattgagtacgtgcgaaccctgggaaccgttgttttggtcggtctgcccgccggcgccgtctgcaagtctcccatcttccagcaggtggctcgatctatccagatcaagggctcttacgttggaaaccgagccgactcccaggaggccattgagttcttctcccgaggtctcgtcaagtcgcccatcatcatcatcggtctgtccgagctggaaaaggtctacaagcttatggaggagggcaagattgccggccgatacgttctggacacctccaagtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 21 - DNA, 6478 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatccacaatgtctgctcccgtcatccccaagacccagaagggtgtcatcttcgagacctccggcggtcctctcatgtacaaggacatccccgtgcctgtgcctgccgacgacgagattctggtcaacgtcaagttctccggagtctgccacacggatctgcacgcctggaagggcgactggcctctggacaccaagcttcctctggtcggaggccacgagggtgccggagtggttgttgccaagggtaagaacgttgacacgtttgagattggcgactatgccggcatcaagtggatcaacaaggcctgctacacctgcgagttctgccaggtggccgccgagcccaactgtcccaacgctaccatgtctggatacacccacgacggctctttccagcagtacgccaccgccaacgccgtgcaggccgcgcacattcccaagaactgcgatctcgccgagattgcccccattctgtgcgccggaatcaccgtctacaaggctctcaagactgccgccatcctcgctggccagtgggttgccgttactggtgctggaggaggactcggaacacttgctgtccagtacgccaaggccatgggctaccgagtgctggccattgacactggcgccgacaaggagaagatgtgcaaggaccttggtgccgaggttttcatcgactttgccaagaccaaggacctcgtcaaggacgtccaggaggccaccaagggcggaccccacgccgtcatcaatgtgtctgtctccgagtttgcagtcaaccagtccattgagtacgtgcgaaccctgggaaccgttgttttggtcggtctgcccgccggcgccgtctgcaagtctcccatcttccagcaggtggctcgatctatccagatcaagggctcttacgttggaaaccgagccgactcccaggaggccattgagttcttctcccgaggtctcgtcaagtcgcccatcatcatcatcggtctgtccgagctggaaaaggtctacaagcttatggaggagggcaagattgccggccgatacgttctggacacctccaagtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctag

SEQ ID NO: 22 - DNA, 7283 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatctcacaagtgagtttgcccacataggtacagtcccagactactcctgaaacacagaggtagatgcccagaccttcactttgaccgctacacaaacaattgcgcagagtgtcgtgccctgtatcacagtctgcacagtgccctgcttgtgtgcatcgcattgcattgattctgtagctctccgcagggagtatttccccatataccaatgctaacacagtgagcgacgttcccaagacacaaaaggccgtcgttttcgaggaagtcaacggacctttgatgtacaaggacattcccgtccccactcccgccaaggacgagctgctcgtcaaggtgcagtattccggtgtctgccactcggatctgtccatctggaagggtgattgggcacagcagctgcggttcagccccaagatgccgctggtcggcggtcatgagggagcaggagaggttgtgggcatgggcgatcaggtgaccggatggcaggtcggagaccgaaccggagtcaagtttatttctggctcttgtctcacttgcgagcactgttctgctggctgggaccagcactgcgtagcccccggcgtgtcaggtctgctcaaagacggctctttccagcagtacgcctgcgtgaaggccgccaccgcaccccgaatccccgattcttgcgatctggctggtgttgcacccgttctgtgtgcaggcatcaccgcctacactgccctcaagaactctggtctcaaggccggtgagtgggtggtgatcaccggagctggaggaggactcggatcctacgccgtccagtacgccaagtgcatgggtttccgtgtgattgccattgacactggagacgacaaggagacccacaccaaggagctgggagccgaggtgtttattgactttgccaagagtggtgctggcatgattgctgagattcacaagctcaccggaggtggcgcccacgccgtggtcaactttgctgtgcaggacgcggctgtcgaggctgccactctgtacgtgcgaacccgaggcactctggttctgtgtgctctgccacccaacggtaccgtcaagagtcacattctcaaccacgtgggtcgaggactcaccatcaagggcagttatgtgggtaataagctggatactcaggaagccattgacttctatgcacggggtctcgtcaagaccaagtaccgtctcggcgagctgagcaagctcgaggagtattaccagcagatgcttgatggtaagattgttggtcgtgtcgttgttgataacagcaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 23 - DNA, 6699 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatctcacaagtgagtttgcccacataggtacagtcccagactactcctgaaacacagaggtagatgcccagaccttcactttgaccgctacacaaacaattgcgcagagtgtcgtgccctgtatcacagtctgcacagtgccctgcttgtgtgcatcgcattgcattgattctgtagctctccgcagggagtatttccccatataccaatgctaacacagtgagcgacgttcccaagacacaaaaggccgtcgttttcgaggaagtcaacggacctttgatgtacaaggacattcccgtccccactcccgccaaggacgagctgctcgtcaaggtgcagtattccggtgtctgccactcggatctgtccatctggaagggtgattgggcacagcagctgcggttcagccccaagatgccgctggtcggcggtcatgagggagcaggagaggttgtgggcatgggcgatcaggtgaccggatggcaggtcggagaccgaaccggagtcaagtttatttctggctcttgtctcacttgcgagcactgttctgctggctgggaccagcactgcgtagcccccggcgtgtcaggtctgctcaaagacggctctttccagcagtacgcctgcgtgaaggccgccaccgcaccccgaatccccgattcttgcgatctggctggtgttgcacccgttctgtgtgcaggcatcaccgcctacactgccctcaagaactctggtctcaaggccggtgagtgggtggtgatcaccggagctggaggaggactcggatcctacgccgtccagtacgccaagtgcatgggtttccgtgtgattgccattgacactggagacgacaaggagacccacaccaaggagctgggagccgaggtgtttattgactttgccaagagtggtgctggcatgattgctgagattcacaagctcaccggaggtggcgcccacgccgtggtcaactttgctgtgcaggacgcggctgtcgaggctgccactctgtacgtgcgaacccgaggcactctggttctgtgtgctctgccacccaacggtaccgtcaagagtcacattctcaaccacgtgggtcgaggactcaccatcaagggcagttatgtgggtaataagctggatactcaggaagccattgacttctatgcacggggtctcgtcaagaccaagtaccgtctcggcgagctgagcaagctcgaggagtattaccagcagatgcttgatggtaagattgttggtcgtgtcgttgttgataacagcaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctag

SEQ ID NO: 24 - DNA, 8181 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaagcttatgcctctactcgactctctcgactttattgttctggtgctggtgggcgtggccaccctggcctttttcaccaagggcaagttgtgggccaaggagcccgagacggacccctatgcaggtggtctgggctcgcagggcttcggatccaccacctcgttcggatcgttcggaggcaactccaacaagacccgagacattaccaagaagctggagcagaccggcaagaacgtgatcatcttctacggctcgcagaccggaactgccgaggattacgccaaccgactgtccaaggaagcaacccagcgatatggcctcaagtccatgaccgccgatctcgaggactacgactacgagaacctcaactcactgggcgacgacattgtcgtgggttttgtcatggccacttacggcgagggagagcccaccgataacgctgtcaacttctacggattcatcaacgacggctcttccgagtgggccgaatccgacgagccctctgccgaccccgactctcccctgtcttctctcaactacgtcattttcggtcttggaaacaacacctacgaacactacaacgagattggccgaaacctggacaagcgactcaagaagctgggcgccaagcgaattggtgactacggcgagggtgacgatggacagggcaccatggaagaggactacctcgcctggaaggacgaccttttctccgcctggaaggaggccaagggtctggacgagcatgaggccaagtatgagccctccgtcaagatctccgagaccggcgagaccggctcttccgaggactcctcttctgttgctgagcctgatgctgaggccatgtctgtgtacctgggtgagcctaacaagaagattctccgaggcgagatcaagggcccctacaacgccggtaaccccttcttggctaacgtttccgagacccgagagctgttccacgaccccaagcgatcctgtatccacgtcgagtttgatgttggcaccaacgtcaagtacaccaccggtgaccatcttgctctgcacattcagaactccgacgaagaagttgagcgattcctcaaggtcattggtctctgggacaagcgacacaatgtcatcaaggccaagcccattgatcccgcctacaagccctctcttcctgtccctactacctatgatactgttgtccgatactacctggagatcaatggtgctgtttcccgacagctgctggccttcattgcccctttcgcccccaccgagactgctaagaaggaggctctgcgacttggttccgacaagaacgcttttgccgatgaggttgccaagcactacaccaacattgcccatgttctctccaagctgtctggcgacgagccttggaccaacgtgcccttctccttcctggttgagtctctcccccatctgatcccccgatactactccatctcgtcctcttccttggtggacaagtccaagatctccatcaccgccgtggtcgagtcccttgaggcccccgagtacgccatcaagggtgttgccaccaacctgctgcttgacatgaagatcaagaaggatggtgttgacccctcaaagtcaaaggacccccaagccgtgcactacgagctgagtggtccccgaggcaagttttggggccacaagctccccgtgcataccagacagtctaacttcaagctgccctctgaccccaagaagcccatcattatgattggtccaggaactggtcttgctcccttccgagcctttgtcatggagcgagctaagcaggccgaaagcggcaccgacgtgggtcaacagcttctcttctttggctgccgaaaccccaacgaggatttcatctacaaggagcagtgggccggcattgagaaggagctcggtgacaagttcaccatggtcactgctttctcccgagtcgaccccgtccaaaaggtctatgtccaacaccgaatgcaggaatatgccaagcagatcaacgatctcatgcaacagggcgcctacttttacgtgtgtggagacgcctcgcgaatggcccgagaggttcaggccaccctggccaagattctgtctgatcagcggggcattcccctgtcttctgctgagcagctggtcaagagcctcaaggtgcagaacgtctaccaggaagatgtgtggtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 25 - DNA, 7597 bp, Artificial sequence
  vector
atcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaagcttatgcctctactcgactctctcgactttattgttctggtgctggtgggcgtggccaccctggcctttttcaccaagggcaagttgtgggccaaggagcccgagacggacccctatgcaggtggtctgggctcgcagggcttcggatccaccacctcgttcggatcgttcggaggcaactccaacaagacccgagacattaccaagaagctggagcagaccggcaagaacgtgatcatcttctacggctcgcagaccggaactgccgaggattacgccaaccgactgtccaaggaagcaacccagcgatatggcctcaagtccatgaccgccgatctcgaggactacgactacgagaacctcaactcactgggcgacgacattgtcgtgggttttgtcatggccacttacggcgagggagagcccaccgataacgctgtcaacttctacggattcatcaacgacggctcttccgagtgggccgaatccgacgagccctctgccgaccccgactctcccctgtcttctctcaactacgtcattttcggtcttggaaacaacacctacgaacactacaacgagattggccgaaacctggacaagcgactcaagaagctgggcgccaagcgaattggtgactacggcgagggtgacgatggacagggcaccatggaagaggactacctcgcctggaaggacgaccttttctccgcctggaaggaggccaagggtctggacgagcatgaggccaagtatgagccctccgtcaagatctccgagaccggcgagaccggctcttccgaggactcctcttctgttgctgagcctgatgctgaggccatgtctgtgtacctgggtgagcctaacaagaagattctccgaggcgagatcaagggcccctacaacgccggtaaccccttcttggctaacgtttccgagacccgagagctgttccacgaccccaagcgatcctgtatccacgtcgagtttgatgttggcaccaacgtcaagtacaccaccggtgaccatcttgctctgcacattcagaactccgacgaagaagttgagcgattcctcaaggtcattggtctctgggacaagcgacacaatgtcatcaaggccaagcccattgatcccgcctacaagccctctcttcctgtccctactacctatgatactgttgtccgatactacctggagatcaatggtgctgtttcccgacagctgctggccttcattgcccctttcgcccccaccgagactgctaagaaggaggctctgcgacttggttccgacaagaacgcttttgccgatgaggttgccaagcactacaccaacattgcccatgttctctccaagctgtctggcgacgagccttggaccaacgtgcccttctccttcctggttgagtctctcccccatctgatcccccgatactactccatctcgtcctcttccttggtggacaagtccaagatctccatcaccgccgtggtcgagtcccttgaggcccccgagtacgccatcaagggtgttgccaccaacctgctgcttgacatgaagatcaagaaggatggtgttgacccctcaaagtcaaaggacccccaagccgtgcactacgagctgagtggtccccgaggcaagttttggggccacaagctccccgtgcataccagacagtctaacttcaagctgccctctgaccccaagaagcccatcattatgattggtccaggaactggtcttgctcccttccgagcctttgtcatggagcgagctaagcaggccgaaagcggcaccgacgtgggtcaacagcttctcttctttggctgccgaaaccccaacgaggatttcatctacaaggagcagtgggccggcattgagaaggagctcggtgacaagttcaccatggtcactgctttctcccgagtcgaccccgtccaaaaggtctatgtccaacaccgaatgcaggaatatgccaagcagatcaacgatctcatgcaacagggcgcctacttttacgtgtgtggagacgcctcgcgaatggcccgagaggttcaggccaccctggccaagattctgtctgatcagcggggcattcccctgtcttctgctgagcagctggtcaagagcctcaaggtgcagaacgtctaccaggaagatgtgtggtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctag

SEQ ID NO: 26 - DNA, 6367 bp, Artificial sequence
  vector
aatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctagataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctag

SEQ ID NO: 27 - DNA, 6951 bp, Artificial sequence
  vector
aatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 28 - DNA, 6379 bp, Artificial sequence
  vector
aatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgtcctgggaaacaatcactcctcctacgccaatcgatacgtttgacagcaacttgcaacgtcttcgagactctttcgagaccggcaagctcgactctgtcgactaccgtctcgagcagctgcgaaccctgtggttcaagttctacgacaacctcgacaacatctacgaggcggtcaccaaggatctccatcgacccaggttcgaaaccgagctcaccgaggtactgtttgttcgagacgagttctccaccgtcatcaagaacctgcgaaagtgggtcaaggaagaaaaggtggagaaccccggaggccccttccagtttgccaacccccgaatccgacccgttcctctgggagtggtgctggtcatcactccctggaactaccccgtcatgctcaacatctcacctgtgattgccgccattgctgccggctgtcccatcgtgctcaagatgtccgagctgtctccccacacttccgctgttcttggccgaatcttcaaggaggccctggaccccggtatcatccaggttgtttacggaggtgtccccgagaccaccgcccttcttacccagcattgggacaagatcatgtacaccggaaacggagccgttggtcgaatcatcgcccaggccgcggtcaagaacctgactcctctagctcttgagcttggtggcaagtcacccgtgttcatcacttccaactgcaagagcgttatgacggccgctcggcgaatcgtgtggggcaagtttgtcaacgccggccagatctgtgtcgctccagactacattctggttgctcccgaaaaggaggccgagctcgtcgcttgtatcaaggaggtgctccaagaacgatacggctccaagagagacgcccaccaccccgatctgtcccatatcatttccaagccccattggaagcgtattcacaacatgatcgcccagaccaagggagacatccaggtgggtggactcgagaacgccgacgaagaccaaaagttcatccagcccacaatcgtctccaacgttccagatgacgacattctcatgcaggacgagattttcggacccatcatccccatcatcaagccccgaaccctcggccagcaggttgattacgtcacaagaaaccatgacacccccctggccatgtacatcttctctgacgaccccaaggaggtggactggctacagacccgaatccgagctggttctgtaaacatcaacgaggtcattgagcaggtcggactggcctctctgcctctcagtggagttggagcttccggaaccggagcataccatggaaaattctccttcgatgtcttcacccacaagcaggccgttatgggacagcccacctggcccttctttgaatacctcatgtattaccggtaccctccttactccgagtacaagatgaaggtgctccgaaccctgttcccaccggttctgattcctcgaaccggccgacccgacgctactgttcttcagcgagttctcggcaacaagctgctttggatcattattgccgcccttgttgcgtacgccaaacgaaatgagctgctcatcaccattgctcagattatgtcggtgtttattaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctagataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctag

SEQ ID NO: 29 - DNA, 6963 bp, Artificial sequence
  vector
aatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgtcctgggaaacaatcactcctcctacgccaatcgatacgtttgacagcaacttgcaacgtcttcgagactctttcgagaccggcaagctcgactctgtcgactaccgtctcgagcagctgcgaaccctgtggttcaagttctacgacaacctcgacaacatctacgaggcggtcaccaaggatctccatcgacccaggttcgaaaccgagctcaccgaggtactgtttgttcgagacgagttctccaccgtcatcaagaacctgcgaaagtgggtcaaggaagaaaaggtggagaaccccggaggccccttccagtttgccaacccccgaatccgacccgttcctctgggagtggtgctggtcatcactccctggaactaccccgtcatgctcaacatctcacctgtgattgccgccattgctgccggctgtcccatcgtgctcaagatgtccgagctgtctccccacacttccgctgttcttggccgaatcttcaaggaggccctggaccccggtatcatccaggttgtttacggaggtgtccccgagaccaccgcccttcttacccagcattgggacaagatcatgtacaccggaaacggagccgttggtcgaatcatcgcccaggccgcggtcaagaacctgactcctctagctcttgagcttggtggcaagtcacccgtgttcatcacttccaactgcaagagcgttatgacggccgctcggcgaatcgtgtggggcaagtttgtcaacgccggccagatctgtgtcgctccagactacattctggttgctcccgaaaaggaggccgagctcgtcgcttgtatcaaggaggtgctccaagaacgatacggctccaagagagacgcccaccaccccgatctgtcccatatcatttccaagccccattggaagcgtattcacaacatgatcgcccagaccaagggagacatccaggtgggtggactcgagaacgccgacgaagaccaaaagttcatccagcccacaatcgtctccaacgttccagatgacgacattctcatgcaggacgagattttcggacccatcatccccatcatcaagccccgaaccctcggccagcaggttgattacgtcacaagaaaccatgacacccccctggccatgtacatcttctctgacgaccccaaggaggtggactggctacagacccgaatccgagctggttctgtaaacatcaacgaggtcattgagcaggtcggactggcctctctgcctctcagtggagttggagcttccggaaccggagcataccatggaaaattctccttcgatgtcttcacccacaagcaggccgttatgggacagcccacctggcccttctttgaatacctcatgtattaccggtaccctccttactccgagtacaagatgaaggtgctccgaaccctgttcccaccggttctgattcctcgaaccggccgacccgacgctactgttcttcagcgagttctcggcaacaagctgctttggatcattattgccgcccttgttgcgtacgccaaacgaaatgagctgctcatcaccattgctcagattatgtcggtgtttattaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 30 - DNA, 6607 bp, Artificial sequence
  vector
aatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcacacaatgtctgacgacaagcacactttcgactttatcattgtcggtggaggaaccgccggccccactctcgcccggcgactggccgatgcctggatctccggtaagaagctcaaggtgctcctgctcgagtccggcccctcttccgagggtgttgatgatattcgatgccccggtaactgggtcaacaccatccactccgagtacgactggtcctacgaggtcgacgagccttacctgtctactgatggcgaggagcgacgactctgtggtatcccccgaggccattgtctgggtggatcctcttgtctgaacacctctttcgtcatccgaggaacccgaggtgatttcgaccgaatcgaagaggagaccggcgctaagggctggggttgggatgatctgttcccctacttccgaaagcacgagtgttacgtgccccagggatctgcccacgagcccaagctcattgacttcgacacctacgactacaagaagttccacggtgactctggtcctatcaaggtccagccttacgactacgcgcccatctccaagaagttctctgagtctctggcttctttcggctacccttataaccccgagatcttcgtcaacggaggagccccccagggttggggtcacgttgttcgttccacctccaacggtgttcgatccaccggctacgacgctcttgtccacgcccccaagaacctcgacattgtgactggccacgctgtcaccaagattctctttgagaagatcggtggcaagcagaccgccgttggtgtcgagacctacaaccgagctgccgaggaggctggccctacctacaaggcccgatacgaggtggttgtgtgctgcggctcttatgcctctccccagcttctgatggtttccggtgttggacccaagaaggagctcgaggaggttggtgtcaaggacatcattttggactctccttacgttggaaagaacctgcaggaccatcttatctgcggtatctttgtcgaaattaaggagcccggatacacccgagaccaccagttcttcgacgacgagggactcgacaagtccaccgaggagtggaagaccaagcgaaccggtttcttctccaatcctccccagggcattttctcttacggccgaatcgacaacctgctcaaggatgatcccgtctggaaggaggcctgcgagaagcagaaggctctcaaccctcgacgagaccccatgggtaacgatccctctcagccccatttcgagatctggaatgctgagctctacattgagctagagatgacccaggctcccgacgagggccagtccgtcatgaccgtcatcggtgagattcttcctcctcgatccaagggttacgtcaagctgctgtcgcccgaccctatggagaaccccgagattgtccacaactacctgcaggaccctgttgacgctcgagtcttcgctgccatcatgaagcacgccgccgacgttgccaccaacggtgctggcaccaaggacctcgtcaaggctcgatggcccccggagtccaagcccttcgaggaaatgtccatcgaggaatgggagacttacgtccgagacaagtctcacacctgtttccacccctgtggtactgtcaagcttggtggtgctaatgataaggaggccgttgttgacgagcgactccgagtcaagggtgtcgacggtctgcgagttgccgacgtctctgtccttccccgagtccccaacggacacacccaggcttttgcctacgctgttggtgagaaggctgccgacctcatccttgccgacattgctggaaaggatctccgacctcgaatctaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctagataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctag

SEQ ID NO: 31 - DNA, 7191 bp, Artificial sequence
  vector
aatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcacacaatgtctgacgacaagcacactttcgactttatcattgtcggtggaggaaccgccggccccactctcgcccggcgactggccgatgcctggatctccggtaagaagctcaaggtgctcctgctcgagtccggcccctcttccgagggtgttgatgatattcgatgccccggtaactgggtcaacaccatccactccgagtacgactggtcctacgaggtcgacgagccttacctgtctactgatggcgaggagcgacgactctgtggtatcccccgaggccattgtctgggtggatcctcttgtctgaacacctctttcgtcatccgaggaacccgaggtgatttcgaccgaatcgaagaggagaccggcgctaagggctggggttgggatgatctgttcccctacttccgaaagcacgagtgttacgtgccccagggatctgcccacgagcccaagctcattgacttcgacacctacgactacaagaagttccacggtgactctggtcctatcaaggtccagccttacgactacgcgcccatctccaagaagttctctgagtctctggcttctttcggctacccttataaccccgagatcttcgtcaacggaggagccccccagggttggggtcacgttgttcgttccacctccaacggtgttcgatccaccggctacgacgctcttgtccacgcccccaagaacctcgacattgtgactggccacgctgtcaccaagattctctttgagaagatcggtggcaagcagaccgccgttggtgtcgagacctacaaccgagctgccgaggaggctggccctacctacaaggcccgatacgaggtggttgtgtgctgcggctcttatgcctctccccagcttctgatggtttccggtgttggacccaagaaggagctcgaggaggttggtgtcaaggacatcattttggactctccttacgttggaaagaacctgcaggaccatcttatctgcggtatctttgtcgaaattaaggagcccggatacacccgagaccaccagttcttcgacgacgagggactcgacaagtccaccgaggagtggaagaccaagcgaaccggtttcttctccaatcctccccagggcattttctcttacggccgaatcgacaacctgctcaaggatgatcccgtctggaaggaggcctgcgagaagcagaaggctctcaaccctcgacgagaccccatgggtaacgatccctctcagccccatttcgagatctggaatgctgagctctacattgagctagagatgacccaggctcccgacgagggccagtccgtcatgaccgtcatcggtgagattcttcctcctcgatccaagggttacgtcaagctgctgtcgcccgaccctatggagaaccccgagattgtccacaactacctgcaggaccctgttgacgctcgagtcttcgctgccatcatgaagcacgccgccgacgttgccaccaacggtgctggcaccaaggacctcgtcaaggctcgatggcccccggagtccaagcccttcgaggaaatgtccatcgaggaatgggagacttacgtccgagacaagtctcacacctgtttccacccctgtggtactgtcaagcttggtggtgctaatgataaggaggccgttgttgacgagcgactccgagtcaagggtgtcgacggtctgcgagttgccgacgtctctgtccttccccgagtccccaacggacacacccaggcttttgcctacgctgttggtgagaaggctgccgacctcatccttgccgacattgctggaaaggatctccgacctcgaatctaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcccggggtggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctag

SEQ ID NO: 32 - DNA, 4852 bp, Artificial sequence
  LEU ADH2 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctagatcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatccacaatgtctgctcccgtcatccccaagacccagaagggtgtcatcttcgagacctccggcggtcctctcatgtacaaggacatccccgtgcctgtgcctgccgacgacgagattctggtcaacgtcaagttctccggagtctgccacacggatctgcacgcctggaagggcgactggcctctggacaccaagcttcctctggtcggaggccacgagggtgccggagtggttgttgccaagggtaagaacgttgacacgtttgagattggcgactatgccggcatcaagtggatcaacaaggcctgctacacctgcgagttctgccaggtggccgccgagcccaactgtcccaacgctaccatgtctggatacacccacgacggctctttccagcagtacgccaccgccaacgccgtgcaggccgcgcacattcccaagaactgcgatctcgccgagattgcccccattctgtgcgccggaatcaccgtctacaaggctctcaagactgccgccatcctcgctggccagtgggttgccgttactggtgctggaggaggactcggaacacttgctgtccagtacgccaaggccatgggctaccgagtgctggccattgacactggcgccgacaaggagaagatgtgcaaggaccttggtgccgaggttttcatcgactttgccaagaccaaggacctcgtcaaggacgtccaggaggccaccaagggcggaccccacgccgtcatcaatgtgtctgtctccgagtttgcagtcaaccagtccattgagtacgtgcgaaccctgggaaccgttgttttggtcggtctgcccgccggcgccgtctgcaagtctcccatcttccagcaggtggctcgatctatccagatcaagggctcttacgttggaaaccgagccgactcccaggaggccattgagttcttctcccgaggtctcgtcaagtcgcccatcatcatcatcggtctgtccgagctggaaaaggtctacaagcttatggaggagggcaagattgccggccgatacgttctggacacctccaagtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 33 - DNA, 4268 bp, Artificial sequence
  URA ADH2 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctagatcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatccacaatgtctgctcccgtcatccccaagacccagaagggtgtcatcttcgagacctccggcggtcctctcatgtacaaggacatccccgtgcctgtgcctgccgacgacgagattctggtcaacgtcaagttctccggagtctgccacacggatctgcacgcctggaagggcgactggcctctggacaccaagcttcctctggtcggaggccacgagggtgccggagtggttgttgccaagggtaagaacgttgacacgtttgagattggcgactatgccggcatcaagtggatcaacaaggcctgctacacctgcgagttctgccaggtggccgccgagcccaactgtcccaacgctaccatgtctggatacacccacgacggctctttccagcagtacgccaccgccaacgccgtgcaggccgcgcacattcccaagaactgcgatctcgccgagattgcccccattctgtgcgccggaatcaccgtctacaaggctctcaagactgccgccatcctcgctggccagtgggttgccgttactggtgctggaggaggactcggaacacttgctgtccagtacgccaaggccatgggctaccgagtgctggccattgacactggcgccgacaaggagaagatgtgcaaggaccttggtgccgaggttttcatcgactttgccaagaccaaggacctcgtcaaggacgtccaggaggccaccaagggcggaccccacgccgtcatcaatgtgtctgtctccgagtttgcagtcaaccagtccattgagtacgtgcgaaccctgggaaccgttgttttggtcggtctgcccgccggcgccgtctgcaagtctcccatcttccagcaggtggctcgatctatccagatcaagggctcttacgttggaaaccgagccgactcccaggaggccattgagttcttctcccgaggtctcgtcaagtcgcccatcatcatcatcggtctgtccgagctggaaaaggtctacaagcttatggaggagggcaagattgccggccgatacgttctggacacctccaagtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 34 - DNA, 5073 bp, Artificial sequence
  LEU ADH5 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctagatcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatctcacaagtgagtttgcccacataggtacagtcccagactactcctgaaacacagaggtagatgcccagaccttcactttgaccgctacacaaacaattgcgcagagtgtcgtgccctgtatcacagtctgcacagtgccctgcttgtgtgcatcgcattgcattgattctgtagctctccgcagggagtatttccccatataccaatgctaacacagtgagcgacgttcccaagacacaaaaggccgtcgttttcgaggaagtcaacggacctttgatgtacaaggacattcccgtccccactcccgccaaggacgagctgctcgtcaaggtgcagtattccggtgtctgccactcggatctgtccatctggaagggtgattgggcacagcagctgcggttcagccccaagatgccgctggtcggcggtcatgagggagcaggagaggttgtgggcatgggcgatcaggtgaccggatggcaggtcggagaccgaaccggagtcaagtttatttctggctcttgtctcacttgcgagcactgttctgctggctgggaccagcactgcgtagcccccggcgtgtcaggtctgctcaaagacggctctttccagcagtacgcctgcgtgaaggccgccaccgcaccccgaatccccgattcttgcgatctggctggtgttgcacccgttctgtgtgcaggcatcaccgcctacactgccctcaagaactctggtctcaaggccggtgagtgggtggtgatcaccggagctggaggaggactcggatcctacgccgtccagtacgccaagtgcatgggtttccgtgtgattgccattgacactggagacgacaaggagacccacaccaaggagctgggagccgaggtgtttattgactttgccaagagtggtgctggcatgattgctgagattcacaagctcaccggaggtggcgcccacgccgtggtcaactttgctgtgcaggacgcggctgtcgaggctgccactctgtacgtgcgaacccgaggcactctggttctgtgtgctctgccacccaacggtaccgtcaagagtcacattctcaaccacgtgggtcgaggactcaccatcaagggcagttatgtgggtaataagctggatactcaggaagccattgacttctatgcacggggtctcgtcaagaccaagtaccgtctcggcgagctgagcaagctcgaggagtattaccagcagatgcttgatggtaagattgttggtcgtgtcgttgttgataacagcaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 35 - DNA, 4489 bp, Artificial sequence
  URA ADH5 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctagatcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacggatctcacaagtgagtttgcccacataggtacagtcccagactactcctgaaacacagaggtagatgcccagaccttcactttgaccgctacacaaacaattgcgcagagtgtcgtgccctgtatcacagtctgcacagtgccctgcttgtgtgcatcgcattgcattgattctgtagctctccgcagggagtatttccccatataccaatgctaacacagtgagcgacgttcccaagacacaaaaggccgtcgttttcgaggaagtcaacggacctttgatgtacaaggacattcccgtccccactcccgccaaggacgagctgctcgtcaaggtgcagtattccggtgtctgccactcggatctgtccatctggaagggtgattgggcacagcagctgcggttcagccccaagatgccgctggtcggcggtcatgagggagcaggagaggttgtgggcatgggcgatcaggtgaccggatggcaggtcggagaccgaaccggagtcaagtttatttctggctcttgtctcacttgcgagcactgttctgctggctgggaccagcactgcgtagcccccggcgtgtcaggtctgctcaaagacggctctttccagcagtacgcctgcgtgaaggccgccaccgcaccccgaatccccgattcttgcgatctggctggtgttgcacccgttctgtgtgcaggcatcaccgcctacactgccctcaagaactctggtctcaaggccggtgagtgggtggtgatcaccggagctggaggaggactcggatcctacgccgtccagtacgccaagtgcatgggtttccgtgtgattgccattgacactggagacgacaaggagacccacaccaaggagctgggagccgaggtgtttattgactttgccaagagtggtgctggcatgattgctgagattcacaagctcaccggaggtggcgcccacgccgtggtcaactttgctgtgcaggacgcggctgtcgaggctgccactctgtacgtgcgaacccgaggcactctggttctgtgtgctctgccacccaacggtaccgtcaagagtcacattctcaaccacgtgggtcgaggactcaccatcaagggcagttatgtgggtaataagctggatactcaggaagccattgacttctatgcacggggtctcgtcaagaccaagtaccgtctcggcgagctgagcaagctcgaggagtattaccagcagatgcttgatggtaagattgttggtcgtgtcgttgttgataacagcaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 36 - DNA, 4742 bp, Artificial sequence
  LEU FALDH3 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctagaatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 37 - DNA, 4158 bp, Artificial sequence
  URA FALDH3 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctagataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctagaatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 38 - DNA, 4754 bp, Artificial sequence
  LEU FALDH4 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctagaatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgtcctgggaaacaatcactcctcctacgccaatcgatacgtttgacagcaacttgcaacgtcttcgagactctttcgagaccggcaagctcgactctgtcgactaccgtctcgagcagctgcgaaccctgtggttcaagttctacgacaacctcgacaacatctacgaggcggtcaccaaggatctccatcgacccaggttcgaaaccgagctcaccgaggtactgtttgttcgagacgagttctccaccgtcatcaagaacctgcgaaagtgggtcaaggaagaaaaggtggagaaccccggaggccccttccagtttgccaacccccgaatccgacccgttcctctgggagtggtgctggtcatcactccctggaactaccccgtcatgctcaacatctcacctgtgattgccgccattgctgccggctgtcccatcgtgctcaagatgtccgagctgtctccccacacttccgctgttcttggccgaatcttcaaggaggccctggaccccggtatcatccaggttgtttacggaggtgtccccgagaccaccgcccttcttacccagcattgggacaagatcatgtacaccggaaacggagccgttggtcgaatcatcgcccaggccgcggtcaagaacctgactcctctagctcttgagcttggtggcaagtcacccgtgttcatcacttccaactgcaagagcgttatgacggccgctcggcgaatcgtgtggggcaagtttgtcaacgccggccagatctgtgtcgctccagactacattctggttgctcccgaaaaggaggccgagctcgtcgcttgtatcaaggaggtgctccaagaacgatacggctccaagagagacgcccaccaccccgatctgtcccatatcatttccaagccccattggaagcgtattcacaacatgatcgcccagaccaagggagacatccaggtgggtggactcgagaacgccgacgaagaccaaaagttcatccagcccacaatcgtctccaacgttccagatgacgacattctcatgcaggacgagattttcggacccatcatccccatcatcaagccccgaaccctcggccagcaggttgattacgtcacaagaaaccatgacacccccctggccatgtacatcttctctgacgaccccaaggaggtggactggctacagacccgaatccgagctggttctgtaaacatcaacgaggtcattgagcaggtcggactggcctctctgcctctcagtggagttggagcttccggaaccggagcataccatggaaaattctccttcgatgtcttcacccacaagcaggccgttatgggacagcccacctggcccttctttgaatacctcatgtattaccggtaccctccttactccgagtacaagatgaaggtgctccgaaccctgttcccaccggttctgattcctcgaaccggccgacccgacgctactgttcttcagcgagttctcggcaacaagctgctttggatcattattgccgcccttgttgcgtacgccaaacgaaatgagctgctcatcaccattgctcagattatgtcggtgtttattaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 39 - DNA, 4170 bp, Artificial sequence
  URA FALDH4 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctagataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctagaatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcccacaatgtcctgggaaacaatcactcctcctacgccaatcgatacgtttgacagcaacttgcaacgtcttcgagactctttcgagaccggcaagctcgactctgtcgactaccgtctcgagcagctgcgaaccctgtggttcaagttctacgacaacctcgacaacatctacgaggcggtcaccaaggatctccatcgacccaggttcgaaaccgagctcaccgaggtactgtttgttcgagacgagttctccaccgtcatcaagaacctgcgaaagtgggtcaaggaagaaaaggtggagaaccccggaggccccttccagtttgccaacccccgaatccgacccgttcctctgggagtggtgctggtcatcactccctggaactaccccgtcatgctcaacatctcacctgtgattgccgccattgctgccggctgtcccatcgtgctcaagatgtccgagctgtctccccacacttccgctgttcttggccgaatcttcaaggaggccctggaccccggtatcatccaggttgtttacggaggtgtccccgagaccaccgcccttcttacccagcattgggacaagatcatgtacaccggaaacggagccgttggtcgaatcatcgcccaggccgcggtcaagaacctgactcctctagctcttgagcttggtggcaagtcacccgtgttcatcacttccaactgcaagagcgttatgacggccgctcggcgaatcgtgtggggcaagtttgtcaacgccggccagatctgtgtcgctccagactacattctggttgctcccgaaaaggaggccgagctcgtcgcttgtatcaaggaggtgctccaagaacgatacggctccaagagagacgcccaccaccccgatctgtcccatatcatttccaagccccattggaagcgtattcacaacatgatcgcccagaccaagggagacatccaggtgggtggactcgagaacgccgacgaagaccaaaagttcatccagcccacaatcgtctccaacgttccagatgacgacattctcatgcaggacgagattttcggacccatcatccccatcatcaagccccgaaccctcggccagcaggttgattacgtcacaagaaaccatgacacccccctggccatgtacatcttctctgacgaccccaaggaggtggactggctacagacccgaatccgagctggttctgtaaacatcaacgaggtcattgagcaggtcggactggcctctctgcctctcagtggagttggagcttccggaaccggagcataccatggaaaattctccttcgatgtcttcacccacaagcaggccgttatgggacagcccacctggcccttctttgaatacctcatgtattaccggtaccctccttactccgagtacaagatgaaggtgctccgaaccctgttcccaccggttctgattcctcgaaccggccgacccgacgctactgttcttcagcgagttctcggcaacaagctgctttggatcattattgccgcccttgttgcgtacgccaaacgaaatgagctgctcatcaccattgctcagattatgtcggtgtttattaagtagcctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 40 - DNA, 4982 bp, Artificial sequence
  LEU FAO1 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctagaatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcacacaatgtctgacgacaagcacactttcgactttatcattgtcggtggaggaaccgccggccccactctcgcccggcgactggccgatgcctggatctccggtaagaagctcaaggtgctcctgctcgagtccggcccctcttccgagggtgttgatgatattcgatgccccggtaactgggtcaacaccatccactccgagtacgactggtcctacgaggtcgacgagccttacctgtctactgatggcgaggagcgacgactctgtggtatcccccgaggccattgtctgggtggatcctcttgtctgaacacctctttcgtcatccgaggaacccgaggtgatttcgaccgaatcgaagaggagaccggcgctaagggctggggttgggatgatctgttcccctacttccgaaagcacgagtgttacgtgccccagggatctgcccacgagcccaagctcattgacttcgacacctacgactacaagaagttccacggtgactctggtcctatcaaggtccagccttacgactacgcgcccatctccaagaagttctctgagtctctggcttctttcggctacccttataaccccgagatcttcgtcaacggaggagccccccagggttggggtcacgttgttcgttccacctccaacggtgttcgatccaccggctacgacgctcttgtccacgcccccaagaacctcgacattgtgactggccacgctgtcaccaagattctctttgagaagatcggtggcaagcagaccgccgttggtgtcgagacctacaaccgagctgccgaggaggctggccctacctacaaggcccgatacgaggtggttgtgtgctgcggctcttatgcctctccccagcttctgatggtttccggtgttggacccaagaaggagctcgaggaggttggtgtcaaggacatcattttggactctccttacgttggaaagaacctgcaggaccatcttatctgcggtatctttgtcgaaattaaggagcccggatacacccgagaccaccagttcttcgacgacgagggactcgacaagtccaccgaggagtggaagaccaagcgaaccggtttcttctccaatcctccccagggcattttctcttacggccgaatcgacaacctgctcaaggatgatcccgtctggaaggaggcctgcgagaagcagaaggctctcaaccctcgacgagaccccatgggtaacgatccctctcagccccatttcgagatctggaatgctgagctctacattgagctagagatgacccaggctcccgacgagggccagtccgtcatgaccgtcatcggtgagattcttcctcctcgatccaagggttacgtcaagctgctgtcgcccgaccctatggagaaccccgagattgtccacaactacctgcaggaccctgttgacgctcgagtcttcgctgccatcatgaagcacgccgccgacgttgccaccaacggtgctggcaccaaggacctcgtcaaggctcgatggcccccggagtccaagcccttcgaggaaatgtccatcgaggaatgggagacttacgtccgagacaagtctcacacctgtttccacccctgtggtactgtcaagcttggtggtgctaatgataaggaggccgttgttgacgagcgactccgagtcaagggtgtcgacggtctgcgagttgccgacgtctctgtccttccccgagtccccaacggacacacccaggcttttgcctacgctgttggtgagaaggctgccgacctcatccttgccgacattgctggaaaggatctccgacctcgaatctaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 41 - DNA, 4398 bp, Artificial sequence
  URA FAO1 cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctagataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctagaatcgatagagaccgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatcacacaatgtctgacgacaagcacactttcgactttatcattgtcggtggaggaaccgccggccccactctcgcccggcgactggccgatgcctggatctccggtaagaagctcaaggtgctcctgctcgagtccggcccctcttccgagggtgttgatgatattcgatgccccggtaactgggtcaacaccatccactccgagtacgactggtcctacgaggtcgacgagccttacctgtctactgatggcgaggagcgacgactctgtggtatcccccgaggccattgtctgggtggatcctcttgtctgaacacctctttcgtcatccgaggaacccgaggtgatttcgaccgaatcgaagaggagaccggcgctaagggctggggttgggatgatctgttcccctacttccgaaagcacgagtgttacgtgccccagggatctgcccacgagcccaagctcattgacttcgacacctacgactacaagaagttccacggtgactctggtcctatcaaggtccagccttacgactacgcgcccatctccaagaagttctctgagtctctggcttctttcggctacccttataaccccgagatcttcgtcaacggaggagccccccagggttggggtcacgttgttcgttccacctccaacggtgttcgatccaccggctacgacgctcttgtccacgcccccaagaacctcgacattgtgactggccacgctgtcaccaagattctctttgagaagatcggtggcaagcagaccgccgttggtgtcgagacctacaaccgagctgccgaggaggctggccctacctacaaggcccgatacgaggtggttgtgtgctgcggctcttatgcctctccccagcttctgatggtttccggtgttggacccaagaaggagctcgaggaggttggtgtcaaggacatcattttggactctccttacgttggaaagaacctgcaggaccatcttatctgcggtatctttgtcgaaattaaggagcccggatacacccgagaccaccagttcttcgacgacgagggactcgacaagtccaccgaggagtggaagaccaagcgaaccggtttcttctccaatcctccccagggcattttctcttacggccgaatcgacaacctgctcaaggatgatcccgtctggaaggaggcctgcgagaagcagaaggctctcaaccctcgacgagaccccatgggtaacgatccctctcagccccatttcgagatctggaatgctgagctctacattgagctagagatgacccaggctcccgacgagggccagtccgtcatgaccgtcatcggtgagattcttcctcctcgatccaagggttacgtcaagctgctgtcgcccgaccctatggagaaccccgagattgtccacaactacctgcaggaccctgttgacgctcgagtcttcgctgccatcatgaagcacgccgccgacgttgccaccaacggtgctggcaccaaggacctcgtcaaggctcgatggcccccggagtccaagcccttcgaggaaatgtccatcgaggaatgggagacttacgtccgagacaagtctcacacctgtttccacccctgtggtactgtcaagcttggtggtgctaatgataaggaggccgttgttgacgagcgactccgagtcaagggtgtcgacggtctgcgagttgccgacgtctctgtccttccccgagtccccaacggacacacccaggcttttgcctacgctgttggtgagaaggctgccgacctcatccttgccgacattgctggaaaggatctccgacctcgaatctaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 42 - DNA, 5971 bp, Artificial sequence
  LEU CPR cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgcctgagtcatcatttatttaccagttggccacaaacccttgacgatctcgtatgtcccctccgacatactcccggccggctgggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgttcgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcacccgagtcctcaaggacgagttcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcaggatctgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcgaattcgtataacttcgtatagcaggagttatccgaagcgataattaccctgttatccctagatcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaagcttatgcctctactcgactctctcgactttattgttctggtgctggtgggcgtggccaccctggcctttttcaccaagggcaagttgtgggccaaggagcccgagacggacccctatgcaggtggtctgggctcgcagggcttcggatccaccacctcgttcggatcgttcggaggcaactccaacaagacccgagacattaccaagaagctggagcagaccggcaagaacgtgatcatcttctacggctcgcagaccggaactgccgaggattacgccaaccgactgtccaaggaagcaacccagcgatatggcctcaagtccatgaccgccgatctcgaggactacgactacgagaacctcaactcactgggcgacgacattgtcgtgggttttgtcatggccacttacggcgagggagagcccaccgataacgctgtcaacttctacggattcatcaacgacggctcttccgagtgggccgaatccgacgagccctctgccgaccccgactctcccctgtcttctctcaactacgtcattttcggtcttggaaacaacacctacgaacactacaacgagattggccgaaacctggacaagcgactcaagaagctgggcgccaagcgaattggtgactacggcgagggtgacgatggacagggcaccatggaagaggactacctcgcctggaaggacgaccttttctccgcctggaaggaggccaagggtctggacgagcatgaggccaagtatgagccctccgtcaagatctccgagaccggcgagaccggctcttccgaggactcctcttctgttgctgagcctgatgctgaggccatgtctgtgtacctgggtgagcctaacaagaagattctccgaggcgagatcaagggcccctacaacgccggtaaccccttcttggctaacgtttccgagacccgagagctgttccacgaccccaagcgatcctgtatccacgtcgagtttgatgttggcaccaacgtcaagtacaccaccggtgaccatcttgctctgcacattcagaactccgacgaagaagttgagcgattcctcaaggtcattggtctctgggacaagcgacacaatgtcatcaaggccaagcccattgatcccgcctacaagccctctcttcctgtccctactacctatgatactgttgtccgatactacctggagatcaatggtgctgtttcccgacagctgctggccttcattgcccctttcgcccccaccgagactgctaagaaggaggctctgcgacttggttccgacaagaacgcttttgccgatgaggttgccaagcactacaccaacattgcccatgttctctccaagctgtctggcgacgagccttggaccaacgtgcccttctccttcctggttgagtctctcccccatctgatcccccgatactactccatctcgtcctcttccttggtggacaagtccaagatctccatcaccgccgtggtcgagtcccttgaggcccccgagtacgccatcaagggtgttgccaccaacctgctgcttgacatgaagatcaagaaggatggtgttgacccctcaaagtcaaaggacccccaagccgtgcactacgagctgagtggtccccgaggcaagttttggggccacaagctccccgtgcataccagacagtctaacttcaagctgccctctgaccccaagaagcccatcattatgattggtccaggaactggtcttgctcccttccgagcctttgtcatggagcgagctaagcaggccgaaagcggcaccgacgtgggtcaacagcttctcttctttggctgccgaaaccccaacgaggatttcatctacaaggagcagtgggccggcattgagaaggagctcggtgacaagttcaccatggtcactgctttctcccgagtcgaccccgtccaaaaggtctatgtccaacaccgaatgcaggaatatgccaagcagatcaacgatctcatgcaacagggcgcctacttttacgtgtgtggagacgcctcgcgaatggcccgagaggttcaggccaccctggccaagattctgtctgatcagcggggcattcccctgtcttctgctgagcagctggtcaagagcctcaaggtgcagaacgtctaccaggaagatgtgtggtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

SEQ ID NO: 43 - DNA, 5387 bp, Artificial sequence
  URA CPR cassette
ggccgcctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagatagagtcgagaattaccctgttatccctacataacttcgtatagcatacattatacgaagttattctgaattccgagaaacacaacaacatgccccattggacagaccatgcggatacacaggttgtgcagtaccatacatactcgatcagacaggtcgtctgaccatcatacaagctgaacagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcggaattcgtataacttcgtatagcaggagttatccgaagcgataattaccctgttatccctagatcgattcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttagctagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaagcttatgcctctactcgactctctcgactttattgttctggtgctggtgggcgtggccaccctggcctttttcaccaagggcaagttgtgggccaaggagcccgagacggacccctatgcaggtggtctgggctcgcagggcttcggatccaccacctcgttcggatcgttcggaggcaactccaacaagacccgagacattaccaagaagctggagcagaccggcaagaacgtgatcatcttctacggctcgcagaccggaactgccgaggattacgccaaccgactgtccaaggaagcaacccagcgatatggcctcaagtccatgaccgccgatctcgaggactacgactacgagaacctcaactcactgggcgacgacattgtcgtgggttttgtcatggccacttacggcgagggagagcccaccgataacgctgtcaacttctacggattcatcaacgacggctcttccgagtgggccgaatccgacgagccctctgccgaccccgactctcccctgtcttctctcaactacgtcattttcggtcttggaaacaacacctacgaacactacaacgagattggccgaaacctggacaagcgactcaagaagctgggcgccaagcgaattggtgactacggcgagggtgacgatggacagggcaccatggaagaggactacctcgcctggaaggacgaccttttctccgcctggaaggaggccaagggtctggacgagcatgaggccaagtatgagccctccgtcaagatctccgagaccggcgagaccggctcttccgaggactcctcttctgttgctgagcctgatgctgaggccatgtctgtgtacctgggtgagcctaacaagaagattctccgaggcgagatcaagggcccctacaacgccggtaaccccttcttggctaacgtttccgagacccgagagctgttccacgaccccaagcgatcctgtatccacgtcgagtttgatgttggcaccaacgtcaagtacaccaccggtgaccatcttgctctgcacattcagaactccgacgaagaagttgagcgattcctcaaggtcattggtctctgggacaagcgacacaatgtcatcaaggccaagcccattgatcccgcctacaagccctctcttcctgtccctactacctatgatactgttgtccgatactacctggagatcaatggtgctgtttcccgacagctgctggccttcattgcccctttcgcccccaccgagactgctaagaaggaggctctgcgacttggttccgacaagaacgcttttgccgatgaggttgccaagcactacaccaacattgcccatgttctctccaagctgtctggcgacgagccttggaccaacgtgcccttctccttcctggttgagtctctcccccatctgatcccccgatactactccatctcgtcctcttccttggtggacaagtccaagatctccatcaccgccgtggtcgagtcccttgaggcccccgagtacgccatcaagggtgttgccaccaacctgctgcttgacatgaagatcaagaaggatggtgttgacccctcaaagtcaaaggacccccaagccgtgcactacgagctgagtggtccccgaggcaagttttggggccacaagctccccgtgcataccagacagtctaacttcaagctgccctctgaccccaagaagcccatcattatgattggtccaggaactggtcttgctcccttccgagcctttgtcatggagcgagctaagcaggccgaaagcggcaccgacgtgggtcaacagcttctcttctttggctgccgaaaccccaacgaggatttcatctacaaggagcagtgggccggcattgagaaggagctcggtgacaagttcaccatggtcactgctttctcccgagtcgaccccgtccaaaaggtctatgtccaacaccgaatgcaggaatatgccaagcagatcaacgatctcatgcaacagggcgcctacttttacgtgtgtggagacgcctcgcgaatggcccgagaggttcaggccaccctggccaagattctgtctgatcagcggggcattcccctgtcttctgctgagcagctggtcaagagcctcaaggtgcagaacgtctaccaggaagatgtgtggtaacctagggtgtctgtggtatctaagctatttatcactctttacaacttctacctcaactatctactttaataaatgaatatcgtttattctctatgattactgtatatgcgttcctctaagacaaatcgaattccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgc

(19) United States
(12) Patent Application Publication
(10) Pub. No.: US 2018/0363011 A1
(43) Pub. Date: Dec. 20, 2018

(54) MICROORGANISMS AND USE THEREOF FOR THE PRODUCTION OF DIACIDS

(71) Applicants: FONDS DE DEVELOPPEMENT DES FILIERES DES OLEAGINEUX ET DES PROTEAGINEUX FIDOP, Paris (FR); INSTITUT NATIONAL DE LA RECHERCHE AGRONOMIQUE (INRA), Paris Cedex 07 (FR)

(72) Inventors: France Thevenieau, Chartres (FR); Jean-Marc Nicaud, Trappes (FR); Vincent Sauveplane, Elancourt (FR); Heber Gamboa-Melendez, Paris (FR); Nicolas Morin, Fontenay-le-Fleury (FR)

(73) Assignees: FONDS DE DEVELOPPEMENT DES FILIERES DES OLEAGINEUX ET DES PROTEAGINEUX FIDOP, Paris (FR); INSTITUT NATIONAL DE LA RECHERCHE AGRONOMIQUE (INRA), Paris Cedex 07 (FR)

(21) Appl. No.: 15/561,753

(22) PCT Filed: Mar. 25, 2016
(86) PCT No.: PCT/EP2016/056693

§ 371 Date: May 4, 2018

(30) Foreign Application Priority Data
Mar. 27, 2015 (EP) 15305452.3

(51) Int. Cl. C12P 7/46; C12N 15/81; C12P 19/34; C12R 1/645

(52) U.S. Cl. CPC C12P 7/46; C12R 1/645; C12P 19/34; C12N 15/815

(57) ABSTRACT

This invention relates to the use of a yeast strain overexpressing at least the following genes: the ALK3 gene, at least one of genes ADH2 and ADH5 and at least one of genes FALDH3 and FALDH4, or gene FA01, for the fermentation-based production of carboxylic diacids.

The present invention relates to microorganisms and use thereof for the production of dicarboxylic acids.

Dicarboxylic acids (also known as “diacids”) are used as starting materials for example in the synthesis of polyamides and polyesters, of lubricant oils, of plasticizers or of fragrances.

The processes for producing diacides vary depending on the number of carbon atoms of the carbon backbone of the diacid in question. For example, azelaic acid (C9 diacid) is conventionally obtained by chemical oxidation of oleic acid with ozone, while sebacic acid (C10 diacid) is produced by alkaline oxidation of ricinoleic acid. Dodecanedioic acid (C12 diacid) is a product of petrochemistry. The microbiological route is used for the production of brassylic acid (C13 diacid) from tridecane.

Given the diversity of the diacids that are used in the various applications, the advantage of a production route that is applicable to the widest possible range of diacids is desirable. Although it is characterized by a slower reaction rate than that of the chemical route, the biological route has the advantage of being applicable to a large variety of substrates.

Although numerous wild-type microbial species, such as Cryptococcus neoformans, Pseudomonas aeruginosa, Candida cloacae, etc., are capable of biosynthesizing diacids, the production levels remain relatively low.

Thus, in order to obtain substantial excretions of diacids, mutants which have been blocked at the level of β-oxidation should be used.

Another, more restrictive, technique using site-directed mutagenesis techniques has been developed for Candida tropicalis. Starting from a wild-type strain belonging to the species, the sequential destruction of the four genes encoding the two acyl-CoA oxidase (Aox) isoenzymes which catalyze the first step of β-oxidation were carried out (Determination of Candida tropicalis Acylcoenzyme A Oxidase Isoenzyme Function by Sequential Gene Disruption. Mol. Cell. Biol. 11, 1991, 4333-4339, and patent U.S. Pat. No. 5 254 466 A1). However the Candida tropicalis strains produced do not appear to be completely stable and lend themselves to possible reversions. It is for this reason that improvements have had to be introduced into the prior art.

Other examples of improvement have been reported. In particular, application WO 2014/100461 relates to biological processes which make it possible to obtain dicarboxylic fatty acids. To do this, some genes of the w-oxidation metabolic pathway were overexpressed, in order to allow the formation of diacids.

However, such methods do not appear to allow optimal production of long-chain diacids.

Thus, a subject of the invention is to overcome the drawbacks of the prior art.

One of the aims of the invention is to provide a process for the synthesis of diacids which allows increased production.

Another aim of the invention is to provide modified microorganisms which make it possible to implement this process.

Yet another aim of the invention is to use novel genetic tools which make it possible to improve the production of diacids by microorganisms.

The invention relates to the use of a yeast strain incapable of degrading fatty acids, in particular a Yarrowia lipolytica strain, overexpressing at least the following genes:
the ALK3 gene, encoding a cytochrome P450 monooxygenase
at least one of the ADH2 and ADH5 genes, each encoding an alcohol dehydrogenase, and
at least one of the FALDH3 and FALDH4 genes, each encoding a fatty aldehyde dehydrogenase, or the FAO1 gene encoding a fatty alcohol oxidase,

for the fermentation-based preparation of at least one dicarboxylic acid from fatty acids or from hydrocarbons, in particular from fatty acids derived from vegetable oils.

The invention is based on the surprising observation made by the inventors that the overexpression of Alk3 genes, of at least one gene chosen from ADH2 and ADH5 and of at least one gene chosen from FALDH3, FALDH4 and FAO1 makes it possible to significantly increase the production of diacids, in particular from fatty acids or from hydrocarbons (alkanes, alkenes or alkynes).

Unexpectedly, the inventors have demonstrated a synergy of the overexpressions of the abovementioned genes on the production of diacids, whereas the simple overexpression of each of these genes has little or no effect or has an opposite effect: the production of diacids is greatly decreased. This result is surprising since it is known from the prior art that, for example, the overexpression of cytochrome P450 can convert fatty acids or hydrocarbons into diacids, without involving other ω-oxidation enzymes, i.e. fatty aldehyde dehydrogenases and fatty alcohol dehydrogenases.

In the invention, the term “diacids” or “dicarboxylic acids” is defined as organic compounds having two carboxyl functions. The molecular formula of these compounds is generally denoted HOOC—R—COOH, where R can be an alkyl, alkenyl, alkynyl or aryl group.

The diacids obtained by means of the process of the invention are derived from linear or branched, saturated or unsaturated hydrocarbons, or from their equivalent carboxylic acids, and are converted via the w-oxidation pathway.

In the invention, the term “overexpression” is intended to mean the level of expression of a gene that has been artificially introduced into the genome of a yeast strain, ectopically or non-ectopically (measured by the amount of RNA produced, or by the amount of protein derived from this RNA), which is at least two times higher than the level of expression of the same endogenous gene. The gene is termed “overexpressed” if the sum of the expressions of the gene (exogenous and optionally endogenous) is at least two times higher than the expression of the endogenous gene when the yeast strain is not transformed or when it is said to be reference wild-type.

In other words, for the example of the ALK3 gene, the yeast strains used in the invention have been previously transformed with a molecule of nucleic acids encoding said ALK3 gene, placed under the control of elements which regulate its expression and which do not correspond to the elements for regulation of the gene in its natural context (for example, a constitutive promoter which is not the endogenous promoter of the ALK3 gene, presence of sequence(s) for increasing or for facilitating the expression—enhancer, etc.). There will be overexpression if the total amount of product expressed by the transformed strain is at least two times higher than the amount of product expressed by a yeast strain not transformed with the ALK3 gene.

Those skilled in the art, with their general knowledge in molecular biology, will be able to quantify this overexpression using quantitative PCR techniques to measure the RNA expression level, or using immunological techniques to measure the amount of proteins.

The example above regarding the ALK3 gene applies, mutatis mutandis, to the other genes overexpressed in the context of the invention.

In order to promote diacid production, it is necessary for the yeast strains used in the context of the invention for carrying out the production process to be incapable of degrading the fatty acids. In other words, it is necessary for the yeast strains used not to be capable of degrading either the fatty acids (carboxylic acids having a long saturated or unsaturated, branched or unbranched carbon-based chain) or the diacids obtained by conversion during the w-oxidation steps.

In the invention, the fatty acids can be considered to be free or in a form which is esterified with glycerol so as to form monoglycerides, diglycerides or triglycerides.

Thus, by limiting fatty acid degradation, and consequently diacid degradation, the latter will accumulate, and their production will thus be increased.

In the invention, use is made of a yeast strain which overexpresses:
the ALK3 gene encoding a cytochrome P450 monooxygenase belonging to the family,
at least one of the ADH2 and ADH5 genes encoding alcohol dehydrogenases, and
at least one of the FALDH3 and FALDH4 genes encoding fatty aldehyde dehydrogenases, and the FAO1 gene encoding a fatty alcohol oxidase. FALDH3 YALI0B01298g is also called HFD4 (Iwama et al., 2014) in Yarrowia lipolytica. FALDH4 YALI0A17875g is also called FALDH1 (Gatter et al., 2014) and HFD3 (Iwama et al., 2014) in Yarrowia lipolytica.

This thus means that, in the invention, the following 21 combinations of genes are envisioned:
ALK3, ADH2 and FALDH2,
ALK3, ADH2 and FALDH4,
ALK3, ADH2 and FAO1,
ALK3, ADH2, FALDH2 and FALDH4,
ALK3, ADH2, FALDH2 and FAO1,
ALK3, ADH2, FALDH4 and FAO1,
ALK3, ADH2, FALDH2, FALDH4 and FAO1,
ALK3, ADH5 and FALDH2,
ALK3, ADH5 and FALDH4,
ALK3, ADH5 and FAO1,
ALK3, ADH5, FALDH2 and FALDH4,
ALK3, ADH5, FALDH2 and FAO1,
ALK3, ADH5, FALDH4 and FAO1,
ALK3, ADH5, FALDH2, FALDH4 and FAO1,
ALK3, ADHA2, ADH5 and FALDH2,
ALK3, ADHA2, ADH5 and FALDH4,
ALK3, ADHA2, ADH5 and FAO1,
ALK3, ADHA2, ADH5, FALDH2 and FALDH4,
ALK3, ADHA2, ADH5, FALDH2 and FAO1,
ALK3, ADHA2, ADH5, FALDH4 and FAO1, and
ALK3, ADHA2, ADH5, FALDH2, FALDH4 and FAO1.

The advantageous yeast strains used in the context of the invention are the following: the strains of Candida spp. yeasts (for example : C. tropicalis, C. viswanathii), the strains of Yarrowia spp. yeasts (in particular Y. lipolytica), the strains of Pichia spp. yeasts, the strains of Saccharomyces spp. yeasts and the strains of Kluyveromyces spp. yeasts.

The advantageous strains according to the invention are Yarrowia lipolytica strains incapable of degrading fatty acids, and which overexpress at least one of the 21 combinations of genes of the invention, listed above.

Advantageously, the ALK3 gene overexpressed in the invention comprises or essentially consists of the nucleic acid sequence SEQ ID NO: 1. ALK3 of the invention can also cover genes having at least 75% identity with the sequence SEQ ID NO: 1, provided that these sequences encode proteins which have a cytochrome P450 monooxygenase acitivity, and in particular the following gene sequences: SEQ ID NO: 2 (YAALOS03-16006g1_1), SEQ ID NO: 3 (YAGA0E09252g1_1) and SEQ ID NO: 4 (YAYAOS2-22892g1_1).

Advantageously, the ADH2 gene overexpressed in the invention comprises or essentially consists of the nucleic acid sequence SEQ ID NO: 5. The ADH2 gene of the invention can also cover genes having at least 75% identity with the sequence SEQ ID NO: 5, provided that these sequences encode proteins which have an alcohol dehydrogenase activity.

In addition, the ADH5 gene overexpressed in the invention comprises or essentially consists of the nucleic acid sequence SEQ ID NO: 6. The ADH5 gene of the invention can also cover genes having at least 80% identity with the sequence SEQ ID NO: 6, provided that these sequences encode proteins which have an alcohol dehydrogenase activity.

Advantageously, the FALDH3 gene overexpressed in the invention comprises or essentially consists of the nucleic acid sequence SEQ ID NO: 7. The FADH3 gene of the invention can also cover genes having at least 80% identity with the sequence SEQ ID NO: 7, provided that these sequences encode proteins which have a fatty aldehyde dehydrogenase activity.

Advantageously, the FALDH4 gene overexpressed in the invention comprises or essentially consists of the nucleic acid sequence SEQ ID NO: 8. The FADH4 gene of the invention can also cover genes having at least 80% identity with the sequence SEQ ID NO: 8, provided that these sequences encode proteins which have a fatty aldehyde dehydrogenase activity.

Advantageously, the FAO1 gene overexpressed in the invention comprises or essentially consists of the nucleic acid sequence SEQ ID NO: 9. The FADH4 gene of the invention can also cover genes having at least 80% identity with the sequence SEQ ID NO: 9, provided that these sequences encode proteins which have a fatty alcohol oxidase activity, and in particular the sequences SEQ ID NO: 10 (YAYA0S1-26698g), SEQ ID NO: 11 (YAGA0F17920g), SEQ ID NO: 12 (YAALOSO4-08768g) and SEQ ID NO: 13 (YAPHOSS-07338g).

The cytochrome P450 monooxygenase activity can be measured by CO Spectrum: differential spectrum between reduced P450 and presence of carbon monoxide and of reduced P450, as described in Estabrook and Werringloer 1978. Methods Enzymol. 52:212-220. Another method consists in placing the enzymes in the presence of substrate (7-ethoxyresorufin, 7-pentoxyresorufin) so that they are metabolized. The reaction product, resorufin, is fluorescent and can be quantified for example using a fluorescence reader.

The fatty aldehyde dehydrogenase activity can for example be measured by studying pyrenedecanal metabolism by HPLC. In the presence of 20 mM sodium pyrophosphate at pH 8, of 1 mM NAD, of Triton X-100 at 1% (v/v; in its reduced form) and of 50 μM of pyrenedecanal, the reaction is carried out in the presence of the enzyme. After reaction at 37° C. for 20-30 min, the reaction is stopped with methanol, and the reaction mixture is centrifuged at 16 000 g before analysis by HPLC.

Another method can be based on Iwama et al., 2014, J. Biol. Cell. n-Decane is added to a cell culture, to a final concentration of 1% for 6 h. The cells are washed, and taken up in a homogenization buffer (25 mM HEPES-NaOH (pH 7.3), 100 mM KCl, 10% glycerol, 1 mM dithiothreitol, and 1% of protease inhibitors) and ground with balls having a diameter of 0.45 to 0.5 mm. The homogenate is centrifuged twice at 1000 g for 10 min at 4° C. 1% v/v of Tween 80 is added to the supernatant, and the mixture is left at 4° C. for 20 min, then centrifuged at 13 000 g for 10 min. The supernatant is then analyzed by mass spectrometry in order to measure the n-decane conversion products.

The alcohol dehydrogenase activity can be measured according to the protocol of Napora-Wijata et al. Biomolecules 2013, 3, 449-460. Briefly, the alcohol dehydrogenase activity is determined by measuring the reduction of NAD(P)+ at 340 nm. 20 μl of solution (alcohol or sugar, 100 mM in 50 mM potassium phosphate, 40 mM KCl, pH 8.5) are added to 140 μl of potassium phosphate (50 mM, 40 mM KCl, pH 8.5), followed by 20 μl of enzyme (in 10 mM sodium phosphate, pH 7.5). The reaction is initiated by adding 20 μl of NAD+ (or NADP+; 10 mM in water) and the reaction is carried out for 10 min. Reactions without substrates are carried out as controls. The activity is defined as the amount of enzyme capable of producing 1 pmol of NADH per min.

The abovementioned yeast strain may be a Yarrowia lipolytica strain transformed such that it overexpresses any one of the combinations of genes mentioned above. In this case, it is an “autologous” overexpression. However, it is possible to transfer the metabolic pathway into another organism, such that the diacid biosynthesis pathway is reproduced. Thus, it is possible to cause a yeast of a genus other than the Yarrowia genus, for example yeasts of the Candida, Pichia or Saccharomyces genera (without being limiting), to overexpress the genes of the abovementioned combinations. This will then be a “heterologous or orthologous” overexpression.

In the invention, the yeast strains used are incapable of degrading fatty acids. This is because the aim of the invention is to increase production of diacids by limiting as much as possible any metabolic pathway of which the aim would be to degrade the biosynthesized diacids. To do this, it is possible,
either to inactivate the degradation pathway: p-oxidation, for example by carrying out a deletion or a disruption of the POX genes encoding the acyl-CoA oxidase isoenzymes, involved in the first step of peroxisomal β-oxidation, in particular the POX1, POX2, POX3, POX4, POX5 and POX6 genes, which will inhibit fatty acid degradation in the peroxisomes,
or to carry out a deletion or a disruption of the MFE2 gene, which is a multifunctional enzyme involved in the second and third steps of peroxisomal β-oxidation,
or to carry out a deletion or a disruption of the FAA1 and/or PXA 1 and/or 2 genes. The FAA1 gene encodes a cytoplasmic fatty acid CoA synthetase and the PXA1 and PXA2 genes encode an ABC transporter involved in fatty acid transport in the peroxisomes.

Thus, in summary, when the modified yeast strain as defined above is used, it is possible to carry out a diacid production by fermentation. The advantageous source of fermentation substrate is a fatty acid, a hydrocarbon or a mixture of fatty acids and hydrocarbons.

If the composition used as substrate comprises several fatty acids or hydrocarbons of different nature (carbon-based chain of different size, presence of unsaturations of substitutions, etc.), the result of the fermentation will result in the obtaining of a mixture of the diacids corresponding to the substrates. For example, if the substrates comprise a C5 hydrocarbon and a C10 hydrocarbon, the result of the fermentation will be the obtaining of a mixture of C5 and C10 diacids. The example above also applies to the carboxylic acids.

In one advantageous embodiment, the invention relates to the use of a Yarrowia lipolytica or Candida tropicalis strain incapable of degrading fatty acids, overexpressing at least the following genes:
the ALK3 gene comprising or consisting of the following sequence SEQ ID NO: 1, encoding a cytochrome P450 monooxygenase, or consisting of a sequence having at least 75% identity with the sequence SEQ ID NO: 1,
at least one of the ADH2 and ADH5 genes comprising or consisting respectively of the sequence SEQ ID NO: 5 and SEQ ID NO: 6, each encoding an alcohol dehydrogenase, and
at least one of the FALDH3 and FALDH4 genes comprising or consisting respectively of the following sequence SEQ ID NO: 7 or SEQ ID NO: 8, each encoding a fatty aldehyde dehydrogenase or the FAO1 gene comprising or consisting of the following sequence SEQ ID NO: 9 encoding a fatty alcohol dehydrogenase,

for the fermentation-based preparation of at least one dicarcarboxylic acid.

Advantageously, the invention relates to the abovementioned use, wherein said yeast strain also overexpresses the CPR1 gene which encodes an NADPH-cytochrome reductase.

Advantageously, in addition to the abovementioned combinations of genes (ALK3, ADH2/5, FALDH3/4 and FAO1), the inventors have shown that the overexpression of the CPR1 gene encoding a cytochrome P450 reductase makes it possible to increase diacid production.

In the invention, the CPR1 gene is defined as comprising or consisting of the nucleic acid sequence SEQ ID NO: 14, or any sequence having at least 80% identity with the sequence SEQ ID NO: 14, provided that these sequences encode a protein having a cytochrome P450 reductase activity.

When the CPR1 gene is overexpressed, the possible strains covered by the invention are the following:
CPR1, ALK3, ADH2 and FALDH2,
CPR1, ALK3, ADH2 and FALDH4,
CPR1, ALK3, ADH2 and FAO1,
CPR1, ALK3, ADH2, FALDH2 and FALDH4,
CPR1, ALK3, ADH2, FALDH2 and FAO1,
CPR1, ALK3, ADH2, FALDH4 and FAO1,
CPR1, ALK3, ADH2, FALDH2, FALDH4 and FAO1,
CPR1, ALK3, ADH5 and FALDH2,
CPR1, ALK3, ADH5 and FALDH4,
CPR1, ALK3, ADH5 and FAO1,
CPR1, ALK3, ADH5, FALDH2 and FALDH4,
CPR1, ALK3, ADH5, FALDH2 and FAO1,
CPR1, ALK3, ADH5, FALDH4 and FAO1,
CPR1, ALK3, ADH5, FALDH2, FALDH4 and FAO1,
CPR1, ALK3, ADHA2, ADH5 and FALDH2,
CPR1, ALK3, ADHA2, ADH5 and FALDH4,
CPR1, ALK3, ADHA2, ADH5 and FAO1,
CPR1, ALK3, ADHA2, ADH5, FALDH2 and FALDH4,
CPR1, ALK3, ADHA2, ADH5, FALDH2 and FAO1,
CPR1, ALK3, ADHA2, ADH5, FALDH4 and FAO1, and
CPR1, ALK3, ADHA2, ADH5, FALDH2, FALDH4 and FAO1.

Advantageously, the invention relates to the abovementioned use, wherein said yeast is also disrupted, or has a deletion for the genes encoding the acyl-CoA oxidase isoenzymes POX1, POX2, POX3, POX4, POX5 and POX6.

As mentioned above, in order to increase diacid production, it is advantageous to limit fatty acid degradation, and in particular degradation by β-oxidation. The concomitant inactivation by deletion or disruption (that is to say the insertion of an element into the sequence of the gene which results in an expression of a nonfunctional product of the gene or in an absence of expression) of the POX1, POX2, POX3, POX4, POX5 and POX6 genes makes it possible to limit or even eliminate this degradation.

The abovementioned deletion or disruption of the POX genes can be carried out as described in international application WO 2006/064131.

It is also possible to use the MTLY66, MTLY81, FT120 and FT130 strains that were deposited with the Collection Nationale de Cultures de Microorganismes [French National Collection of Microorganism Cultures] under the respective registration numbers CNCM 1-3319, CNCM I-3320, CNCM I-3527 and CNCM I-3528.

In another advantageous embodiment, the invention relates to the abovementioned use, wherein said yeast is also disrupted or has a deletion for the DGA1, DGA2 and/or LRO1 genes. In other words, in another advantageous embodiment, the invention relates to the abovementioned use, wherein said yeast is also disrupted or has a deletion for at least one of the DGA1, DGA2 and LRO1 genes.

The technical effect of this disruption is to limit fatty acid storage. Indeed, once produced, fatty acids can be stored and thus escape the conversion into dicarboxylic acids. The pool of stored fatty acids represents from 10% to 70% of the total amount of fatty acids produced or assimilated by a microorganism. Thus, in order to prevent escape from conversion into diacids, and to increase the production of the latter, it is advantageous to limit the storage.

The disruption or deletion of at least one of the DGA1, DGA2 and/or LRO1 genes inhibits said storage.

The term “DGA1, DGA2 and/or LRO1” is intended to mean the following combinations: DGA1 alone, DGA2 alone, LRO1 alone, the combination DGA1 and DGA2, the combination DGA1 and LRO1, the combination DGA2 and LRO1, and the combination DGA1 and DGA2 and LRO1.

The DGA2 gene encodes a diacylglycerol acyl transferase of DGAT1 type, the DGA1 gene encodes a diacylglycerol acyl transferase of DGAT2 type, the LRO1 gene encodes a phospholipid:diacylglycerol acyl transferase involved in the synthesis of triglycerol from diacylglycerol via the independent acetyl CoA pathway.

The DGA1 gene comprises or consists of the sequence SEQ ID NO: 15, the DGA2 gene comprises or consists of the sequence SEQ ID NO: 16 and the LRO1 gene comprises or consists of the sequence SEQ ID NO: 17.

In yet another advantageous embodiment, the invention relates to the abovementioned use, wherein said yeast strain overexpressing said genes is derived from the Yarrowia lipolytica yeast strain OLEO-X.

The OLEO-X yeast strain is itself derived from the w29 strain deposited with the ATCC (American Type Culture Collection) under number ATCC 20460, and has the following genotype: MATA ura-3-302 leu2-270 xpr2-322 pox1-6Δ dga1Δ lro1Δ dga2Δ fad2Δ.

Thus, in one advantageous embodiment, the invention relates to the use of a Yarrowia lipolytica strain of genotype MATA ura-3-302 leu2-270 xpr2-322 pox1-6Δ dga1Δ lro1Δ dga2Δ fad2Δ, overexpressing at least one of the following genes:
the ALK3 gene comprising or consisting of the following sequence SEQ ID NO: 1, encoding a cytochrome P450 monooxygenase, or consisting of a sequence having at least 75% identity with the sequence SEQ ID NO: 1,
at least one of the ADH2 and ADH5 genes comprising or consisting respectively of the sequence SEQ ID NO: 5 and SEQ ID NO: 6, each encoding an alcohol dehydrogenase, and
at least one of the FALDH3 and FALDH4 genes comprising or consisting respectively of the following sequence SEQ ID NO: 7 or SEQ ID NO: 8, each encoding a fatty aldehyde dehydrogenase, or the FAO1 gene, comprising or consisting of the following sequence SEQ ID NO: 9 encoding a fatty alcohol oxidase, optionally also overexpressing the CPR1 gene encoding an NADPH-cytochrome reductase comprising or consisting of the following sequence SEQ ID NO: 14,

for the fermentation-based preparation of at least one dicarboxylic acid, in particular from at least one hydrocarbon or at least one fatty acid.

In the context of the abovementioned use, it is advantageous to have, as bioconversion source, either hydrocarbons, or fatty acids, having a long chain, that is to say having a carbon backbone of more than 10 carbon atoms.

It is in particular advantageous, in order to have diacids exhibiting at least one unsaturation, to use monounsaturated or polyunsaturated fatty acids or hydrocarbons, that is to say those which have at least one carbon-carbon double bond on said carbon backbone.

Advantageously, the invention relates to the use of any one of the following strains:
the Y4832 strain, also called JMY4832, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was filed with the CNCM on Mar. 14, 2016, under number CNCM I-5072,
the Y4833 strain, also called JMY4833, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016, under number CNCM I-5073, and
the Y4834 strain, also called JMY4834, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016, under number CNCM I-5074,

for the fermentation-based preparation of at least one dicarboxylic acid as defined above.

The invention also relates to a method for producing at least one dicarboxylic acid, comprising the following steps:

a) a growth phase, in which is placed in culture a yeast strain incapable of degrading fatty acids, overexpressing at least the following genes:
the ALK3 gene, encoding a cytochrome P450 monooxygenase
at least one of the ADH2 and ADH5 genes, each encoding alcohol dehydrogenases, and
at least one of the FALDH3 or FALDH4 genes, encoding fatty aldehyde dehydrogenases or the FAO1 gene encoding a fatty alcohol oxidase,

in a culture medium consisting essentially of an energy substrate which comprises at least one carbon source and one nitrogen source, and

b) a bioconversion phase, in which said yeast strain is brought into contact with at least one fatty acid, preferably in the presence of an energy substrate.

All the definitions and descriptions relating to the use defined above are applicable, mutatis mutandis, to the process, or the method, mentioned above.

In the process for producing diacids according to the invention, the chosen strain is placed in culture in a medium consisting essentially of an energy substrate which comprises at least one carbon source and one nitrogen source in order to cause said strain to grow. This is the growth phase. This can be important insofar as the incapacity to degrade fatty acids can interfere with yeast growth.

The bioconversion substrate (alkane or mixture of alkanes, fatty acid or mixture of fatty acids, fatty acid ester or mixture of fatty acid esters or natural oil or mixture of these various substrates) is then added so as to initiate the bioconversion into diacids.

During the bioconversion phase, the culture medium can comprise a provision of secondary energy substrate consisting, in general, of at least one polyhydroxylated compound, for instance glycerol or a sugar, including in particular glucose.

The mutant strains that can be used in the process of the invention can be obtained from the Po1d strain, which derives from the Yarrowia lipolytica wild-type strain W29. The Po1d strain is a strain that is auxotrophic for leucine (leu−) and uracil (ura−). It is descrdibed in the review by G. Barth et al.: Yarrowia lipolytica in: Nonconventional Yeasts in Biotechnology A Handbook (Wolf, K., Ed.), Vol. 1, 1996, pp. 313-388. Springer-Verlag, Berlin, Heidelberg, New York. It is listed under CLIB139 in the CLIB.

The principle of the process according to the invention is thus to bioconvert the hydrocarbons into diacids, and the fatty acids into diacids.

For example, octadecane C₁₈F₃₈ will be converted into octadecanedioic acid, just as stearic acid, oleic acid (cis-octadec-9-enoic acid) will be converted into cis-octadec-9-enedioic acid, etc. Those skilled in the art are capable of knowing the diacide obtained from the fatty acid or from the hydrocarbon that is added during the bioconversion step.

Advantageously, the invention relates to the abovementioned process, wherein said yeast strain also overexpresses the CPR1 gene which encodes an NADPH-cytochrome reductase.

Advantageously, the invention relates to a method as defined above, also comprising a step of recovering, isolating or purifying said at least one dicarboxylic acid formed.

Of course, it is advantageous, when the process is carried out, to recover the diacids formed by means of a technique known to those skilled in the art, such as calcium salt precipitation.

In another advantageous embodiment, the invention relats to a method as defined above, in which the fatty acids are in the form of a mixture, and in particular in the form of an oil or of a mixture of alkanes, in particular an oil chosen from:
vegetable oils such as rapeseed oil, oleic rapeseed oil, sunflower oil, oleic sunflower oil, coconut oil, palm oil, palm kernel oil, olive oil, groundnut oil, soybean oil, corn oil, mustard oil, castor oil, palm olein, palm stearin, safflower oil, sesame oil, linseed oil, hazelnut oil, grapeseed oil, hemp oil or a by-product derived from the extraction of said oils, comprising at least 30% of a mixture of fatty acids, for instance esterification liquors, bottoms of tanks, deodorization condensates, washing waters or neutralization pastes,
fish oils, in particular of oily fish, and
microbial oils derived from microorganisms termed oleaginous, that is to say capable of storing fatty acids at more than 20% of their dry weight, derived from yeasts, bacteria or microalgae.

These examples of oils are given by way of indication and could not limit the scope of the invention.

In the invention, the term “vegetable oil” is intended to mean a fatty substance extracted from an oleaginous plant.

The term “oleaginous plant” is intended to mean any plants of which the seeds, nuts or fruits contain lipids.

A fatty substance is a substance composed of molecules having hydrophobic properties. The fatty substances are mainly composed of fatty acids and triglycerides which are esters consisting of a glycerol molecule and of three fatty acids. The other components form what is known as the unsaponifiable material.

The extraction of the vegetable oil by conventional methods often requires various preliminary operations, such as shelling. After these operations, the crop is ground into a paste. The paste, or sometimes the whole fruit, is boiled in the presence of water and with stirring until the oil separates. These conventional methods have a low degree of efficiency.

Modern methods for recovering the oil comprise breaking and pressing steps, and also dissolution in a solvent, usually hexane. The extraction of the oil with a solvent is a more efficient method than pressing. The residue left after the extraction of the oil (oilcake or flour) is used as animal feed.

The crude vegetable oils are obtained without additional treatment other than degumming or filtration. In order to make them suitable for human consumption, edible vegetable oils are refined in order to remove the impurities and toxic substances, a process involving whitening, deodorization and cooling. The vegetable oils envisioned in the invention comprise crude, refined or fractionated oils or the by-products derived from extraction of the oils.

Apart from a few exceptions, and unlike animal fats, vegetable oils contain mainly unsaturated fatty acids of two types: monounsaturated (palmitic acid, oleic acid, erucic acid) and polyunsaturated (linoleic acid).

In another advantageous embodiment, the invention relates to a method as defined above, wherein said yeast is also disrupted for the genes encoding the acyl-CoA oxidase isoenzymes POX1, POX2, POX3, POX4, POX5 and POX6.

In another advantageous embodiment, the invention relates to a method as defined above, wherein said yeast is also disrupted or has a deletion for the DGA1, DGA2 and/or LRO1 genes.

In another advantageous embodiment, the invention relates to a method as defined above, wherein said yeast strain over expressing said genes is derived from the OLEO-X strain.

In yet another advantageous embodiment, the invention relates to a process as defined previously, wherein said diacids are obtained from fatty acids or from hydrocarbons, which are present in the form of a mixture having, by weight, an amount of more than 30% of fatty acids or of hydrocarbons having more than 10 carbon atoms, in particular C₁₄⁻C₂₆ fatty acids or alkanes.

In the invention, the term “at least 30% of fatty acids or of hydrocarbons” is intended to mean an amount of fatty acids or of hydrocarbons of 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% by weight relative to the total weight of the composition.

The term “fatty acid or hydrocarbons having more than 10 atoms” defines linear or branched (CnH2n+2) alkanes, linear or branched (CnH2n) alkenes, or linear or branched (CnH2n−2) alkynes having at least 10 carbon atoms.

The term “C₁₄-C₂₆ fatty acids or hydrocarbons” is intended to mean C14, C15, C16, C17, C18, C19, C20, C21, C22, C23, C24, C25 or C26 fatty acids or hydrocarbons.

In yet another advantageous embodiment, the invention relates to the abovementioned use, wherein said fatty acids or hydrocarbons are present in the form of a mixture having, by weight, an amount of more than 30% of fatty acids having more than 10 carbon atoms, in particular C₁₄-C₂₆ fatty acids or hydrocarbons, and having, by weight, in particular more than 30% of fatty acids or hydrocarbons that are at least monounsaturated.

Even more advantageously, the invention relates to the abovementioned method, said at least one fatty acid being a mixture of fatty acids having, by weight, an amount of more than 30% of oleic acid relative to the total weight of the mixture.

It is advantageous, in order to obtain unsaturated diacids, to use fatty acids derived from vegetable oils, which have one or more unsaturated fatty acids.

In particular, in order to obtain C18 diacids comprising an unsaturation (DC18:1), it is advantageous to use a vegetable oil or a composition comprising an amount of at least 30% of oleic acid of formula I below:

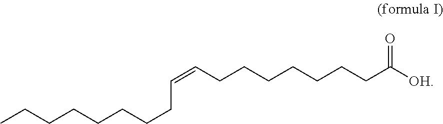 [SMILES: CCCCCCCC/C=C\CCCCCCCC(=O)O]

The advantageous vegetable oils are the following: hazelnut oil which comprises approximately 77% by weight of oleic acid, olive oil which comprises approximately 72% by weight of oleic acid, avocado oil which comprises approximately 68% by weight of oleic acid, rapeseed oil which comprises approximately 56% by weight of oleic acid, oleic sunflower oil which comprises approximately 80% by weight of oleic acid, groundnut oil which comprises approximately 35% by weight of oleic acid, palm olein which comprises approximately 40% by weight of oleic acid, sesame oil which comprises approximately 39% by weight of oleic acid or palm oil which comprises approximately 36% by weight of oleic acid.

The term “approximately X % by weight” is intended to mean the value of X % plus or minus 1% by weight. This approximation is linked to the variability of the methods for measuring the amount of oleic acid contained in an oil, and also the variability of production depending on the plants used.

Advantageously, the invention also relates to a method for producing at least one dicarboxylic acid, in particular cis-octadec-9-enedioic acid, comprising the following steps:

a) a growth phase, in which is placed in culture a Yarrowia lipolytica yeast strain, in particular of genotype MATA ura3-302 leu2-270 xpr2-322 pox1-6Δ dga1Δ lro1Δ dga2Δ fad2Δ, incapable of degrading fatty acids, and optionally of storing fatty acids in the form of triglyceride, overexpressing at least the combinations of genes chosen from the group below:
the ALK3 gene, in particular comprising or consisting of the following sequence SEQ ID NO: 1, or comprising or consisting of a sequence having at least 75% identity with the sequence SEQ ID NO: 1 and having a cytochrome P450 monooxygenase activity, the ADH2 gene comprising or consisting respectively of the sequence SEQ ID NO: 5 or comprising or consisting of a sequence having at least 75% identity with the sequence SEQ ID NO: 5 and having an alcohol dehydrogenase activity and the FALDH3 gene comprising or consisting respectively of the sequence SEQ ID NO: 7 or comprising or consisting of a sequence having at least 75% identity with the sequence SEQ ID NO: 7 and having a fatty aldehyde dehydrogenase activity,
the ALK3 gene, in particular comprising or consisting of the following sequence SEQ ID NO: 1, or comprising or consisting of a sequence having at least 75% identity with the sequence SEQ ID NO: 1 and having a cytochrome P450 monooxygenase activity, the ADH2 gene comprising or consisting respectively of the sequence SEQ ID NO: 5 or comprising or consisting of a sequence having at least 75% identity with the sequence SEQ ID NO: 5 and having an alcohol dehydrogenase activity and the FALDH4 gene comprising or consisting respectively of the sequence SEQ ID NO: 8 or comprising or consisting of a sequence having at least 75% identity with the sequence SEQ ID NO: 8 and having a fatty aldehyde dehydrogenase activity,
the ALK3 gene, in particular comprising or consisting of the following sequence SEQ ID NO: 1, or comprising or consisting of a sequence having at least 75% identity with the sequence SEQ ID NO: 1 and having a cytochrome P450 monooxygenase activity, the ADH2 gene comprising or consisting respectively of the sequence SEQ ID NO: 5 or comprising or consisting of a sequence having at least 75% identity with the sequence SEQ ID NO: 5 and having an alcohol dehydrogenase activity and the FAO1 gene comprising or consisting respectively of the sequence SEQ ID NO: 9 or comprising or consisting of a sequence having at least 75% identity with the sequence SEQ ID NO: 9 and having a fatty alcohol oxidase activity,
the ALK3 gene, in particular comprising or consisting of the following sequence SEQ ID NO: 1, or comprising or consisting of a sequence having at least 75% identity with the sequence SEQ ID NO: 1 and having a cytochrome P450 monooxygenase activity, the ADHS gene comprising or consisting respectively of the sequence SEQ ID NO: 6 or comprising or consisting of a sequence having at least 75% identity with the sequence SEQ ID NO: 6 and having an alcohol dehydrogenase activity and the FALDH3 gene comprising or consisting respectively of the sequence SEQ ID NO: 7 or comprising or consisting of a sequence having at least 75% identity with the sequence SEQ ID NO: 7 and having a fatty aldehyde dehydrogenase activity,
the ALK3 gene, in particular comprising or consisting of the following sequence SEQ ID NO: 1, or comprising or consisting of a sequence having at least 75% identity with the sequence SEQ ID NO: 1 and having a cytochrome P450 monooxygenase activity, the ADH5 gene comprising or consisting respectively of the sequence SEQ ID NO: 6 or comprising or consisting of a sequence having at least 75% identity with the sequence SEQ ID NO: 6 and having an alcohol dehydrogenase activity and the FALDH4 gene comprising or consisting respectively of the sequence SEQ ID NO: 8 or comprising or consisting of a sequence having at least 75% identity with the sequence SEQ ID NO: 8 and having a fatty aldehyde dehydrogenase activity, and
the ALK3 gene, in particular comprising or consisting of the following sequence SEQ ID NO: 1, or comprising or consisting of a sequence having at least 75% identity with the sequence SEQ ID NO: 1 and having a cytochrome P450 monooxygenase activity, the ADH5 gene comprising or consisting respectively of the sequence SEQ ID NO: 6 or comprising or consisting of a sequence having at least 75% identity with the sequence SEQ ID NO: 6 and having an alcohol dehydrogenase activity and the FAO1 gene comprising or consisting respectively of the sequence SEQ ID NO: 9 or comprising or consisting of a sequence having at least 75% identity with the sequence SEQ ID NO: 9 and having a fatty aldehyde dehydrogenase activity,

optionally also overexpressing the CPR1 gene comprising or consisting of the sequence SEQ ID NO: 14 or comprising or consisting of a sequence having at least 80% identity with the sequence SEQ ID NO: 14 and having an NADPH-cytochrome reductase activity,

in a culture medium consisting essentially of an energy substrate which comprises at least one carbon source and one nitrogen source, and

b) a bioconversion phase, in which said yeast strain is brought into contact with an oil, in particular a vegetable oil, such as the abovementioned oils, a fish oil or an oil from yeasts, bacteria or microalgae, preferably in the presence of an energy substrate.

Advantageously, the invention also relates to a method for producing at least one dicarboxylic acid, in particular cis-octadec-9-enedioic acid, comprising the following steps:

a) a growth phase, in which is placed in culture a Yarrowia lipolytica yeast strain chosen from the following strains:
the Y4832 strain, also called JMY4832, is characterized by the genotype MATA ura3-302 leu2-270 xpr2-322 pox1-6≢ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016, under number CNCM I-5072,
the Y4833 strain, also called JMY4833, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016, under number CNCM I-5073, and
the Y4834 strain, also called JMY4834, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016, under number CNCM I-5074,

in a culture medium essentially consisting of an energy substrate which comprises at least one carbon source and one nitrogen source, and

b) a bioconversion phase, in which said yeast strain is brought into contact with an oil, in particular a vegetable oil, such as the abovementioned oils, a fish oil or an oil from yeasts, bacteria or microalgae, preferably in the presence of an energy substrate, and

c) optionally, a step of purifying the diacids obtained.

The invention also relates to a composition comprising a mixture of dicarboxylic acids that can be obtained by means of the process as defined above.

The compositions of diacids obtained according to the abovementioned process will not directly give, by weight, a conversion of the alkanes and fatty acids that will be supplied for carrying out the process.

This is because, during the bioconversion of the hydrocarbons and fatty acids by the modified yeast strains, as defined above, in addition to the synthesis of diacids from the exogenous provision, said strains will be capable of synthesizing their own fatty acids for the formation of their cell membrane. These fatty acids will not be stored or degraded since the yeast strains of the invention are knocked out for these metabolic pathways.

Thus, the fatty acids synthesized by the yeasts during their growth may also be bioconverted into diacids.

Consequently, there is no linearity between the amount of fatty acid provided by a given oil, and the amount of diacids obtained. However, the compositions that can be obtained by means of the process of the invention comprise a high proportion of diacid because of the improved efficiency of the yeast strains used.

Moreover, the invention relates to a composition comprising:
a first nucleic acid molecule corresponding to the ALK3 gene, encoding a cytochrome P450 monooxygenase,
at least one second nucleic acid molecule corresponding to at least one of the ADH2 and ADH5 genes, each encoding alcohol dehydrogenases, and
at least one third nucleic acid molecule corresponding to at least one of the FALDH3 or FALDH4 genes, encoding fatty aldehyde dehydrogenases or corresponding to the FAO1 gene encoding a fatty alcohol oxygenase,

said first nucleic acid molecule, second nucleic acid molecule and third nucleic acid molecule being bonded or individualized.

The abovementioned composition should be understood in the following way:
it comprises either a first nucleic acid molecule corresponding to the ALK3 gene, a second nucleic acid molecule corresponding to the ADH2 gene, or to the ADH5 gene, and a third nucleic acid molecule corresponding to the FALDH3 gene, or to the FALDH4 gene, or to the FAO1 gene,
or a first nucleic acid molecule corresponding to the ALK3 gene, said molecule being fused or bonded to a second nucleic acid molecule corresponding to the ADH2 gene, or to the ADH5 gene, and, independent of the first two, a third nucleic acid molecule corresponding to the FALDH3 gene, or to the FALDH4 gene, or to the FAO1 gene,
or a first nucleic acid molecule corresponding to the ALK3 gene and, independently, a second nucleic acid molecule corresponding to the ADH2 gene, or to the ADH5 gene, said molecule being fused to a third nucleic acid molecule corresponding to the FALDH3 gene, or to the FALDH4 gene, or to the FAO1 gene,
or a first nucleic acid molecule corresponding to the ALK3 gene, said molecule being fused to a third nucleic acid molecule corresponding to the FALDH3 gene, or to the FALDH4 gene, or to the FAO1 gene, and, independently, a second nucleic acid molecule corresponding to the ADH2 gene, or to the ADH5 gene,
or a first nucleic acid molecule, corresponding to the ALK3 gene, said molecule being fused or bonded to a second nucleic acid molecule corresponding to the ADH2 gene, or to the ADH5 gene, said molecule being fused to a third nucleic acid molecule corresponding to the FALDH3 gene, or to the FALDH4 gene, or to the FAO1 gene (that is to say one and the same molecule),

optionally in combination with another nucleic acid molecule corresponding to the CPR1 gene.

Also envisioned are recombinant vectors comprising said nucleic acid molecules, and means allowing the expression of said genes.

Advantageously, the invention relates to a composition as defined above, wherein
the first nucleic acid molecule corresponds to the ALK3 gene, said first nucleic acid molecule essentially comprising or consisting of the sequence SEQ ID NO: 1,
the second nucleic acid molecule corresponds to the ADH2 gene, and essentially comprises or consists of the sequence SEQ ID NO: 5, SEQ ID NO: 32 or SEQ ID NO: 33, or to the ADH5 gene, and essentially comprises or consists of the sequence SEQ ID NO: 6, SEQ ID NO: 34 or SEQ ID NO: 35, and
the third nucleic acid molecule corresponds to the FALDH3 gene, and essentially comprises or consists of the sequence SEQ ID NO: 7, SEQ ID NO: 36 or SEQ ID NO: 37, or to the FALDH4 gene, and essentially comprises or consists of the sequence SEQ ID NO: 8, SEQ ID NO: 38 or SEQ ID NO: 39, or to the FAO1 gene, and essentially comprises or consists of the sequence SEQ ID NO: 9, SEQ ID NO: 40 or SEQ ID NO: 41,

optionally in combination with a fourth nucleic acid molecule sequence corresponding to the CPR1 gene, essentially comprising or consisting of the sequence SEQ ID NO: 14, SEQ ID NO: 42 or SEQ ID NO: 43.

In the case of an abovementioned composition where each of the molecules is cloned into a vector, the composition comprises
the first nucleic acid molecule essentially comprising or consisting of the sequence SEQ ID NO: 18 or SEQ ID NO: 19
the second nucleic acid molecule essentially comprising or consisting of the sequence SEQ ID NO: 20, SEQ ID NO: 21, SEQ ID NO: 22 or SEQ ID NO: 23, and
the third nucleic acid molecule essentially comprising or consisting of the sequence SEQ ID NO: 26, SEQ ID NO: 27, SEQ ID NO: 28, SEQ ID NO: 29, SEQ ID NO: 30 or SEQ ID NO: 31,

optionally in combination with a fourth nucleic acid molecule sequence essentially comprising or consisting of the sequence SEQ ID NO: 24 or SEQ ID NO: 25.

The invention also relates to the various nucleic acid molecules comprising or consisting of the following sequences: SEQ ID NOs: 18 to 31.

The invention relates, in addition, to a yeast strain transformed by a composition comprising at least one nucleic acid molecule as defined above.

The various yeast strains envisioned are those described above.

Advantageously, the invention relates to an abovementioned yeast strain, said yeast being a Yarrowia lipolytica strain.

Moreover, the invention relates to a Yarrowia lipolytica strain chosen from the following strains:
the Y3551 strain, of genotype MATA ura3-302 leu2-270 xpr2-322 pox1-6Δ dga1Δ lro1Δ dga2Δ fad2Δ ALK3-LEU2 and of phenotype [Leu+ Ura−],
the JMY3950 strain, derived from the Y3551 strain, of genotype MATA ura3-302 leu2-270 xpr2-322 pox1-6Δ dga1Δ lro1Δ dga2Δ fad2Δ ALK3-LEU2 CPR1-URA3 and of phenotype [Leu+ Ura+], deposited on Mar. 26, 2015 with the CNCM (Collection Nationale de Culture de microorganismes, [French National Collection of Microorganism Cultures], Institut Pasteur, 25 rue du Docteur Roux, F-75724 PARIS Cedex 15) under number CNCM I-4963,
the Y4428 strain, derived from the JMY3950 strain, of genotype MATA ura3-302 leu2-270 xpr2-322 pox1-6Δ dga1Δ lro1Δ dga2Δ fad2Δ ALK3 CPR1 and of phenotype [Leu− Ura−],
the Y4457 strain, derived from the Y4428 strain, of genotype MATA ura3-302 leu2-270 xpr2-322 pox1-6Δ dga1Δ lro1Δ dga2Δ fad2Δ ALK3 CPR1 ADH2-URA3 and of phenotype [Leu− Ura+], and
the Y4832, Y4833 and Y4834 strains, deposited on Mar. 14, 2016 at the CNCM under the respective numbers CNCM I-5072, CNCM I-5073 and CNCM I-5074, these strains being derived from the Y4457 strain, and having the genotype MATA ura3-302 leu2-270 xpr2-322 pox1-6Δ dga1Δ lro1Δ dga2Δ fad2Δ ALK3 CPR1 ADH2-URA3 FAO1-LEU2 and the phenotype [Leu+ Ura+].

More specifically, the Y4832 strain, also called JMY4832, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016 under number CNCM I-5072.

The Y4833 strain, also called JMY4833, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on March 14, 2016 under number CNCM I-5073.

The Y4834 strain, also called JMY4834, is characterized by the genotype MATA ura3-302 leu2-270 xpr2-322 pox1-6Δ dga1Δ lro1Δ dga2Δ fad2Δ ALK3 CPR1 ADH2-URA3 FAO1-LEU2 and has the phenotype [Leu+ Ura+]. This strain was deposited with the CNCM on Mar. 14, 2016 under number CNCM I-5074.

### FIGURE LEGEND

FIGS. 1A to 1E represent TLC chromatograms obtained for the conversion of C12:0 with microsomes of yeasts transformed with various constructs. P, P1 and P2 represent the reaction products, S represents the substrate. The x-axis represents the mobility in mm, and the y-axis represents the radioactivity in arbitrary units.

FIG. 1A represents the TLC histogram of microsomes of yeasts transformed with an empty vector.

FIG. 1B represents the TLC histogram of microsomes of yeasts transformed with a vector expressing ALK2.

FIG. 1C represents the TLC histogram of microsomes of yeasts transformed with a vector expressing ALK 3.

FIG. 1D represents the TLC histogram of microsomes of yeasts transformed with a vector expressing ALK 5.

FIG. 1E represents the TLC histogram of microsomes of yeasts transformed with a vector expressing ALK 11.

FIGS. 2A to 2C show the conversion of oleic acid in the presence of microsomes expressing Alk3p.

FIG. 2A represents TLC chromatograms of microsomes of yeasts transformed with an empty vector (top panel) or with Alk3p (bottom panel). The x-axis corresponds to the time expressed in minutes. 1 and 2 represent the two conversion products obtained.

FIG. 2B represents a mass spectrum of product 1 observed in FIG. 2A.

FIG. 2C represents a mass spectrum of product 2 observed in FIG. 2A.

FIGS. 3A and 3B show the degree of fatty acid conversion.

FIG. 3A represents a histogram showing the specific activity (in pg/min/mg) of conversion of 100 μM fatty acids, indicated along the x-axis, by microsomes of yeast expressing Alk3p. The gray bars represent the w-oxidation products and the white bars the diacids.

FIG. 3B represents a histogram showing the specific activity (in pg/min/mg) of conversion of 100 μM fatty acids, indicated along the x-axis, by microsomes of yeast expressing Alk5p. The gray bars represent the w-oxidation products and the white bars the diacids.

FIG. 4 represents a graph showing the production over diacid by two OLEOX yeast strains (B and C) overexpressing the ALK3 gene. The production is compared to that obtained by an OLEOX strain not transformed with ALK3 (A). The x-axis represents the culture time in hours and the y-axis represents the amount of DC18:1 in g/l.

FIG. 5 represents a graph showing the production over time of diacid by two OLEOX-CPR1 yeast strains (B and C) overexpressing the FALDH3 gene and FALDH4 gene respectively. The production is compared to that obtained by an OLEOX strain not transformed with either one of the FALDH3 or 4 genes (A). The x-axis represents the culture time in hours and y-axis represents the amount of DC18:1 in g/l.

FIG. 6 represents a graph showing the production over time of diacid by two OLEOX yeast strains overexpressing the CPR1+ALK3+ADH2+FALDH3 genes (curve with triangles) and CPR1+ALK3+ADH2+FALDH4 genes (curve with squares). The production is compared to that obtained by an OLEOX strain overexpressing only CPR1 (curve with open squares). The x-axis represents the culture time in hours and the y-axis represents the amount of DC18:1 in g/l.

FIG. 7 represents a graph showing the production over time of diacid by three OLEOX yeast strains overexpressing the CPR1+ALK3+ADH2+FAO1 genes (A, B and C). The production is compared to that obtained by an OLEOX strain overexpressing only CPR1 (D). The x-axis represents the culture time in hours and the y-axis represents the amount of DC18:1 in g/l.

FIG. 8 represents a graph showing the productivity of three OLEOX yeast strains overexpressing the CPR1+ALK3+ADH2+FAO1 genes (A, B and C). The production is compared to that obtained by an OLEOX strain overexpressing only CPR1 (D). The x-axis represents the culture time in hours and the y-axis represents the amount of DC18:1 in g/l.

FIG. 9 represents a graph showing the productivity of three yeast strains overexpressing the CPR1+ADH2 genes (B) or the CPR1+ADH5 genes (C). The production is compared to that obtained by a strain only CPR1 (A). The x-axis represents the culture time in hours and the y-axis represents the amount of DC18:1 in g/l.

### EXAMPLES

### Example 1—Process for Producing Dicarboxylic Acids from Oleic Sunflower Oil with a Strain According to the Invention

A preculture of the strain, stored on agar medium having the composition: yeast extract 10 g/l; peptone 10 g/l; glucose 10 g/l; agar 20 g/l is prepared using an inoculation which gives an initial absorbance of the preculture medium of around 0.30. The preculture is carried out with orbital shaking (200 rpm) for 24 h at 30° C. in a 500 ml flange flask containing 25 ml of medium (10 g/l of yeast extract; 10 g/l of peptone; 20 g/l of glucose).

The medium used for the culture is composed of deionized water, yeast extract at 10 g/l; tryptone at 20 g/l; glucose at 40 g/l and oleic sunflower oil at 30 g/l.

The inoculation of the fermenter is carried out with the entire preculture flask.

The culture is carried out at 30° C. in a 4 l fermenter with 2 l of medium at an aeration rate of 0.5 vvm and a shaking speed of 800 rpm provided by a dual-effect centripetal turbine.

After 17 hours of culture, as soon as the glucose of the medium is exhausted, 60 ml of oleic sunflower oil are added to the reactor which is subjected to a continuous feed of glycerol in a proportion of 1 ml/h. The pH of the culture is then maintained in a range of 7.5 to 8 by regulated addition of 4 M sodium hydroxide. The fermentation lasts 130 h. At the end of culture, the cell biomass is removed by centrifugation. The supernatant is then acidified to pH 2.5 by addition of 6M HCl and the insoluble dicarboxylic acids are recovered by centrifugation of the acidified must and then dried.

The dicarboxylic acid composition of the mixture is determined by gas chromatography on a DB1 column after conversion of the dicarboxylic acids to diesters according to the method described by Uchio et al., Agr Biol Chem 36, No. 3, 1972, 426-433. The temperature of the chromatograph oven is programmed from 150° C. to 280° C. at a rate of 8° C. per min.

### Example 2—Alk3p Converts the Fatty Acids to Diacids In Vitro

In Yarrowia lipolytica, there are 17 genes encoding cytochromes P450, 12 of which belong to the CYP52 family. All these CYP52 genes are inducible in the presence of alkanes.

It has been shown that their deletion affects yeast growth in the presence of alkanes.

The inventors thus tested the function of 7 of these Yarrowia lipolytica genes in order to determine their biological role in aliphatic molecule metabolism.

### Results

The enzymological studies on the members of the CYP52 family were carried out by studying lauric acid metabolism.

In order to confirm their hypotheses according to which these enzymes catalyze fatty acids by oxygenation reaction, the inventors performed a screening using microsomes of S. cerevisiae WAT11 yeast transformed with 6 of the genes of the CYP52 family, cloned into Yarrowia lipolytica: ALK2, ALK3, ALK4, ALK5, ALK6 and ALK11.

The incubations were carried out on the model substrate constituted by lauric acid (C12.0), in the presence of NADPH.

The reaction mixtures were deposited on TLC plates, and then developed and analyzed.

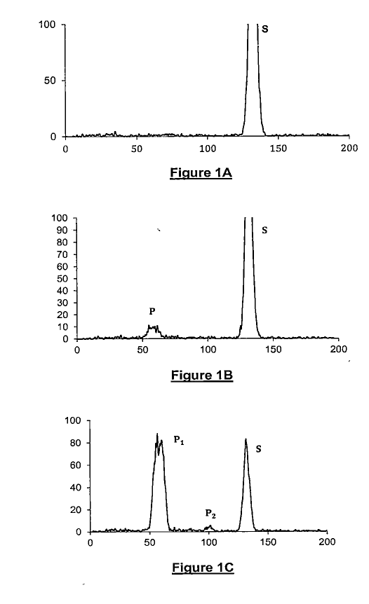

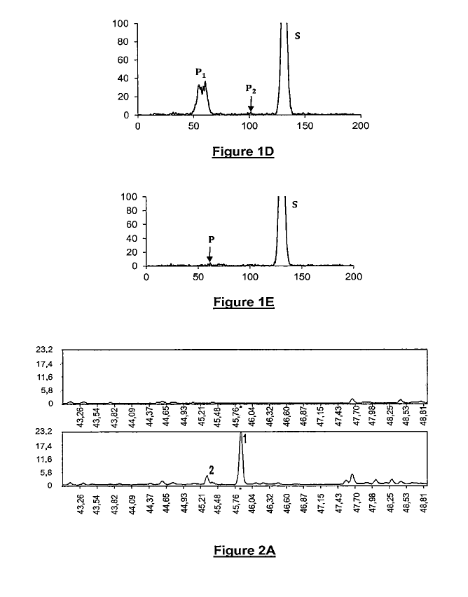

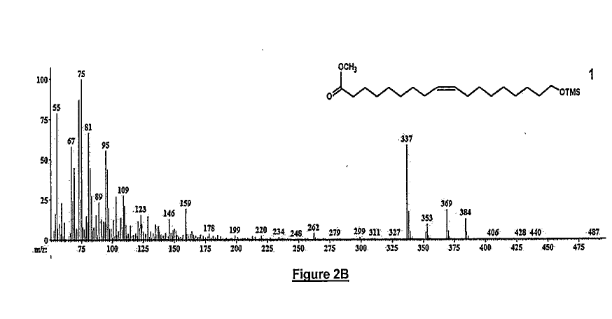

As shown in FIGS. 1A to 1E, only four of the 6 microsomal preparations are capable of converting lauric acid into a highly polar product. No peak is observed for the control (reaction without NADPH), with the exception of the peak corresponding to the substrate. The results indicate that the Alk2p, Alk3p, Alk5p and Alk11p proteins are capable of metabolizing lauric acid.

In order to study the substrate specificity of these enzymes, the inventors incubated each microsomal preparation with free fatty acids of different size and at various levels of unsaturation (for example, myristic acid—C14:0, palmitic acid—C16:0, stearic acid—C18:0, oleic acid—C18:1 and linoleic acid—C18:2).

The degree of conversion for these substrates incubated at a concentration of 100 μM shows that most of the fatty acids are converted:

| | Degree of conversion (%) | | | | | |
|---|---|---|---|---|---|---|
| Gene | C_{12:0} | C_{14:0} | C_{16:0} | C_{18:0} | C_{18:1} | C_{18:2} |
| ALK2 | 7.3 | 1.1 | 0.5 | N.D. | N.D. | N.D. |
| ALK3 | 65 | 68 | 20 | 3 | 27 | 35 |
| ALK4 | N.D. | N.D. | N.D. | N.D. | N.D. | 3 |
| ALK5 | 24 | 27 | 12 | 2 | 13 | 11 |
| ALK6 | N.D. | N.D. | N.D. | N.D. | N.D. | N.D. |
| ALK11 | Traces | N.D. | N.D. | N.D. | 2 | 4 |

ND: not detectable

For example, Alk2p appears to be involved in short-chain fatty acid metabolism, with a degree of conversion which decreases from lauric acid to palmitic acid. No significant conversion of C18:0 is observed with this enzyme.

Alk4p, Alk6p and Alk11p show, for their part, either an absence of activity or a very weak activity.

The microsomes containing Alk3p and Alk5p, for their part, convert all the substrates with a high degree of conversion. Furthermore, Alk3p shows two conversion products for all the substrates except for stearic acid. The first peak has a profile that is expected for an w-hydroxylated fatty acid, while the second appears to correspond to a diacid. The examples of lauric acid conversion are shown in FIGS. 1A to 1E.

In the light of these results, the inventors further studied Alk3p in order to characterize the reaction products.

Preparations of fresh microsomes of yeast expressing Alk3p were carried out. The inventors standardized the total protein content and performed new incubations with lauric acid and palmitic acid and also the abovementioned three C18 fatty acids (stearic acid, oleic acid and linoleic acid). All the reactions were carried out in duplicate in the presence of NADPH for analysis by TLC and GC-MS. The TLC chromatograms clearly show the capacity of Alk3p to convert each of the substrates except for stearic acid into two products. The two products have an w-oxidation and diacid profile as expected. With regard to stearic acid, only an w-oxidation product is obtained. The GC-MS analyses confirm ω-oxidation of all the substrates. However, under the conditions used by the inventors, no diacid is detectable for the fatty acids having less than 18 carbons.

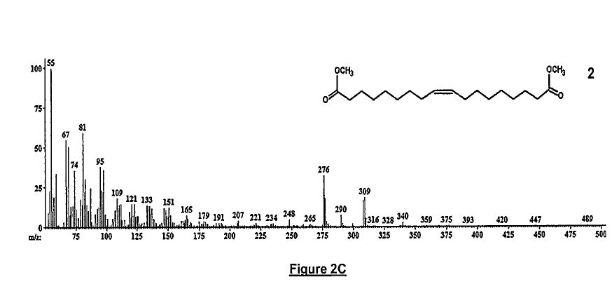

The analyses of the reaction products obtained by virtue of Alk3p are:
for an incubation with lauric acid (C12:0), the mass spectrum obtained for the product peak shows m/Z ions (relative intensity in %) at 73 (43%) (CH₃)₃Si⁺, 75 (50%) [(CH₃)₂Si⁺═O], 103 (20%) [CH₂(OSi(CH₃)₃)], 146 (5%) [CH₂═C⁺(OSi(CH₃)₃—OCH₃], 159 (6%) [CH₃—O⁺═C⁺(OSi(CH₃)₃)CH═CH₂], 255 (100%) (M-47) [loss of methanol for the fragment (M-15)], 271 (5%) (M-31) (loss of OCH₃ of the methyl ester), and 287 (42%) (M-15) (loss of CH₃ of the TMSi group). This fragmentation profile is characteristic of a 12-hydroxylauric acid derivative (M=302 g/mol);
for an incubation with palmitic acid (C16:0), the mass spectrum obtained for the product peak shows m/Z ions (relative intensity in %) at 73 (28%) (CH₃)₃Si⁺, 75 (39%) [(CH₃)₂Si⁺═O], 103 (14%) [CH₂(OSi(CH₃)₃)], 146 (7%) [CH₂═C⁺(OSi(CH₃)₃—OCH₃], 159 (6%) [CH₃—O⁺═C⁺(OSi(CH₃)₃)CH═CH₂], 311 (100%) (M-47) [loss of methanol for the fragment (M-15)], 327 (4%) (M-31) (loss of OCH₃ of the methyl ester), and 343 (32%) (M-15) (loss of CH₃ of the TMSi group). This fragmentation profile is characteristic of a 16-hydroxypalmitic acid (M=358 g/mol);
for an incubation with stearic acid (C18:0), the mass spectrum obtained for the product peak shows m/Z ions (relative intensity in %) at 73 (51%) (CH₃)₃Si⁺, 75 (94%) [(CH₃)₂Si⁺═O], 103 (15%) [CH₂(OSi(CH₃)₃)], 146 (9%) [CH₂═C⁺(OSi(CH₃)₃—OCH₃], 159 (4%) [CH₃—O⁺═C⁺(OSi(CH₃)₃)CH═CH₂], 339 (100%) (M-47) [loss of methanol for the fragment (M-15)], 355 (3%) (M-31) (loss of OCH₃ of the methyl ester), 371 (35%) (M-15) (loss of CH₃ of the TMSi group), and 386 (2%) (M). This fragmentation profile is characteristic of an 18-hydroxystearic acid derivative (M =386 g/mol).
For an incubation with oleic acid (C18:1), two products were detected by GC-MS analysis (FIGS. 2A to 2C). The mass spectrum obtained for the first product peak shows m/Z ions (relative intensity in %) at 73 (86%) (CH₃)₃Si⁺, 75 (100%) [(CH₃)₂Si⁺═O], 103 (26%) [CH₂(OSi(CH₃)₃)], 146 (14%) [CH₂═C⁺(OSi(CH₃)₃—OCH₃], 159 (18%) [CH₃—O⁺═C⁺(OSi(CH₃)₃)CH═CH₂], 337 (60%) (M-47) [loss of methanol for the fragment (M-15)], 353 (8%) (M-31) (loss of OCH₃ of the methyl ester), 369 (19%) (M-15) (loss of CH₃ of the TMSi group), and 384 (12%) (M). This fragmentation profile is characteristic of an 18-hydroxyoleic acid derivative (M=384 g/mol) [30]. The second peak shows m/z ions (relative intensity in %) at 55 (100%), 276 (35%), 290 (8%), 309 (18%) (M-31) (loss of OCH₃ of the methyl ester) and 340 (3%) (M). This fragmentation profile is characteristic of an authentic derivative of 1,18-octadeca-9-enedioic acid (M=340 g/mol).
For an incubation with linoleic acid (C18:2), two products were detected by GC-MS analysis. The mass spectrum obtained for the first product peak shows m/Z ions (relative intensity in %) at 73 (100%) (CH₃)₃Si⁺, 75 (91%) [(CH₃)₂Si⁺═O], 103 (15%) [CH₂(OSi(CH₃)₃)], 146 (7%) [CH₂═C⁺(OSi(CH₃)₃—OCH₃], 159 (8%) [CH₃—O⁺═C⁺(OSi(CH₃)₃)CH═CH₂], 335 (8%) (M-47) [loss of methanol for the fragment (M-15)], 351 (2%) (M-31) (loss of OCH₃ of the methyl ester), 367 (7%) (M-15) (loss of CH₃ of the TMSi group), and 382 (2%) (M). This fragmentation profile is characteristic of an 18-hydroxylinoleic acid derivative (M=382 g/mol). The second peak shows m/z ions (relative intensity in %) at 55 (60%), 274 (12%), 307 (10%) (M-31) (loss of OCH₃ of the methyl ester) and 338 (2%) (M). This fragmentation profile could be that of a 1,18-octadeca-9,12-dienedioic derivative (M=338 g/mol). This hypothesis is also supported by the retention times in the GC-MS analysis system used. Indeed, ΔRTs between the hydroxyls and the diacids for oleic acid are 0.5 minute. The same ΔRT between the hydroxy and the potential diacid is also observed for linoleic acid (RT of the potential 1,18-octadeca-9,12-dienedioic acid is 45.002 min, RT for 18-hydroxylinoleic acid is 45.511 min, RT of 1,18-octadeca-9-enedioic acid is 45.299 min and RT of 18-hydroxy-oleic acid is 45.853 min).

The same results are obtained with the Alk5p protein.

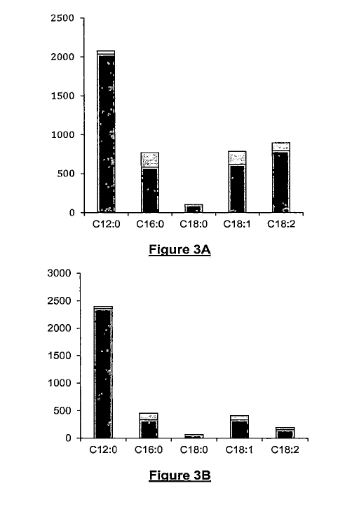

The TLC chromatograms were used to calculate the specific activity of Alk3p and Alk5p for the substrates tested. The results for Alk3p and Alk5p are presented in FIGS. 3A and 3B.

There is no great difference between Alk3p and Alk5p for the various fatty acids tested. However, when looking at the C19 fatty acid profiles, it appears that Alk3p is a better candidate for oxidation of the long chains compared with Alk5p. Furthermore, the conversion of free fatty acid to diacid, catalyzed by Alk3p, is more efficient than with Alk5p.

### CONCLUSION

In Yarrowia lipolytica, ALK gene expression is known to be strongly regulated by alkanes. With regard to their activity in vitro on free fatty acids, one hypothesis is that Alk3p and/or Alk5p could be involved in the successive terminal oxidation of alkanes while successively converting them into fatty alcohol, then fatty acids, then fatty hydroxy alcohols and finally into diacids.

Such conversion could result in substrates that can be used as carbon and energy sources by the β-oxidation pathway.

In the in vitro experiments, the inventors demonstrated that Alk3p and Alk5p efficiently catalyze the ω-oxidation of C₁₂ to C₁₈ free fatty acids.

These studies reveal the great product and substrate diversity of the Alk proteins of Y. lipolytica. By virtue of these results, the inventors now have an indication as to which protein is capable of producing a product of interest. This knowledge is important for creating novel strains capable of bioconversion of oleic acid to its corresponding diacid.

### Materials and Methods

### Cloning of the ALK Gene from Y. lipolytica

The coding sequences of the CYP52 genes were cloned by PCR using a DNA preparation from the Yarrowia lipolytica W29 strain. The sense and antisense primers were prepared by including restriction sites at the two ends in order to carry out the clonings. The PCR amplification was carried out using the Pyrobest polymerase for 30 cycles (15 seconds at 96° C., 30 seconds at 55° C., 1 minute 30 seconds at 72° C.). The resulting DNA fragments were purified by electrophoresis using the QIAquick Gel

Extraction kit. The purified fragments were digested by the appropriate combination of restriction enzymes and ligated into the pYeDP60 shuttle vector using T4 DNA ligase. The ligation products were used to transform E. coli Mach 1T1 made competent chemically. The transformed E. coli cells were selected on LB medium supplemented with 100 μg/ml of ampicillin. The plasmids of a single colony were purified by miniprep.

The integrity of the plasmid and its sequence were validated by restriction analysis and DNA sequencing (GATC Biotech, Constance, Germany).

### Heterologous Expression in S. cerevisiae

The expression of the proteins of the 6 members of the 6YP52 family cloned was carried out using a heterologous system specifically designed for the expression of cytochrome P450 enzymes, based on the pYeDP60 vector and the Saccharomyces cerevisiae WAT11 strain. The WAT11 strain was transformed with each of the pYeDP60 constructs using the lithium acetate LiAc method. The transformants were selected by plating out on YNB plates lacking uracil. The yeasts are left in culture and the expression of cytochrome P450 was induced as described in Pompon et al., 1996. For each transformant, the microsomes were prepared by manually breaking the cells using glass beads (0.45 mm in diameter) in 50 mM of a Tris-HCl buffer (pH 7.5) containing 1 mM EDTA and 600 mM of sorbitol. The homogenate was subjected to centrifugation (10 000 g-15 min) and the resulting supernatant was subjected to ultracentrifugation (100 000 g-1 h). The microsome pellet was resuspended in 50 mM Tris-HCl (pH 7.4), mM EDTA and 30% (v/v) of glycerol with a Potter-Elvehjem homogenizer. The volume of buffer used for resuspending the microsomes was determined by the approximate weight of the wet pellet of yeast obtained after growth (1 ml of buffer per 2 g of cell pellet). The total concentrations of protein in the microsomes were estimated using the Bradford test and homogenized at 15 mg/ml using the appropriate volume of resuspension buffer. The microsome preparation was stored at −20° C. All the experiments for the microsome preparations were carried out between 0 and 4° C.

### In Vitro Enzymatic Assay

The activity of the cytochrome P450 enzymes was evaluated in vitro using various radiolabeled fatty acids. The standard test (0.1 ml) contained 20 ml sodium phosphate (pH 7.4), 1 ml NADPH, a radiolabeled substrate (100 μM) and 0.15 mg of microsomal protein. The reactions were carried out in a waterbath at 27° C. with continuous shaking. The reaction is initiated by adding NADPH and stopped after 20 min by adding 20 μl of acetonitrile containing 0.2% of acetic acid. The reactions were then revealed by direct application of the incubation medium onto the TLC plates or by GC-MS analysis carried out by means of an extraction with organic solvents and a derivation step as described below.

### TLC Analysis

The reaction mixtures were deposited directly on TLC plates covered with silica in order to separate the incubation products from the initial substrate. The plates were developed using an ether/petroleum ether/formic acid mixture (50:50:1, v/v/v). The plates were scanned using a radioactivity detector. The chromatograms resulting from the TLC make it possible to determine the degrees of conversion for each cytochrome P450/fatty acid combination, based on the radioactivity detected by the reader. The mobility of the products on the TLC plate is a good indication of the type of oxygenation reaction that was carried out on the substrate (i.e. hydroxylation, epoxidation, diacid formation). These results were confirmed by GC/MS as far as possible.

### GC-LS Analysis

The metabolites were extracted from the reaction mixture by successive liquid/liquid extractions with diethyl ether and hexane as solvents. The solvents were then evaporated off under a nitrogen stream. The lipids were methylated by means of a reaction in acidic methanol (MeOH/H₂SO₄, 99:1, v/v-1 h-100° C.) and trimethylsilylates with N,O-bistrimethylsilyltrifluoroacetamide containing 1% (v/v) of trimethylchlorosilane. The GC/MS analyses were carried out on a gas chromatograph equipped with a capillary column with an internal diameter of 0.25 mm and a film thickness of 0.25 μm. The gas chromatograph was combined with a selective quadrupole mass detector. The mass spectrum was recorded at 70 eV and analyzed as in Eglinton et al., 1996. The hydroxylated fatty acids just like the dicarboxylic acids formed during the enzymatic reactions were identified by analysis of their mass spectrum and compared with controls when this was necessary.

### Example 3—Overexpression of ALK3

In the light of the results obtained in Example 2, the inventors tested the overexpression of the ALK3 gene in Yarrowia lipolytica with a view to increasing the production of diacid from a source of fatty acid, and in particular of oleic acid.

It had been decided to carry out these modifications in two genetic contexts: 1) the Y2149 production strain (effective strain but which contains the Candida tropicalis CYP51A17 gene and which is not completely blocked for lipid storage in TAG form) and Y2159+CPR1 (derived from the OLEO-X strain which produces slightly fewer diacids, is more sensitive to lipids, but which had the advantage of no longer storing fatty acids in triacylglycerol forms).

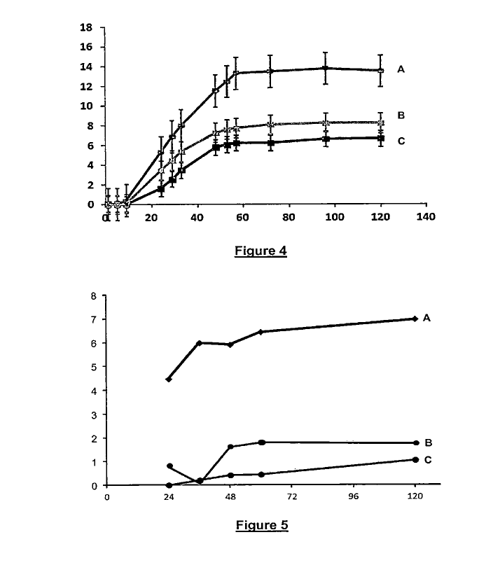

The overexpression of Alk3 and of Cyt B5 in the two genetic contexts did not allow an improvement in the production of cis-octadec-9-enedioic acid (DC18:1); on the contrary, the inventors observed a decrease in DC18:1 production (FIG. 4).

These results are the opposite of the observed effect of an oleic acid-specific hydroxylase activity of ALK3 in vitro in Example 2.

According to these disappointing results, the inventors sought to know whether other enzymes, such as the FALDH enzymes, which are involved in the final step of the diacid synthesis pathway, or other enzymes of the synthesis pathway, such as ADHs and FAO, could be advantageous for increasing diacid production.

### Example 4—Overexpression of the FALDH3 or FALDH4 Genes

In parallel, the inventors identified the genes potentially encoding a fatty aldehyde dehydrogenase activity (four genes known as FALDH1-4). These genes have shown, during the transcriptome analysis during a DC18:1 production time course (DCA7 fermentation), a strong expression during the diacid production phase.

The expression cassettes for the four genes encoding the FALDHs were constructed under the control of the pTEF constitutive promoter. The inventors transformed the two strains: Y2149 and Y2159 (production strain and OLEO-X strain). The strains obtained were verified by PCR and placed in collection.

In order to known whether the final step of the synthesis of DC18:1, which involves the FALDH enzymes, is crucial, the inventors transformed them with the vectors for overexpression in the OLEO-X strain which overexpresses the CPR1 gene.

The inventors obtained only strains which overexpress the FALDH3 and FALDH4 genes. It is possible that the overexpression of the FALDH1 and FALDH2 genes is toxic and lethal to the production strains.

After characterization of the strains, the inventors tested the diacid production by comparing the OLEOX strains overexpressing CPR1 and FALDH3 and the OLEOX strains overexpressing CPR1 and FALDH4. As a control, the OLEOX strain overexpressing only CPR1 was used.

The results are presented in FIG. 5.

The results show that the overexpression of FALDH3 or FALDH4 does not improve DC18:1 production, quite the contrary there is a decrease in diacid production. These results are therefore similar to those observed for the overexpression of ALK3.

### Example 5—Overexpression of the ADH2 and ADH5 Genes

The inventors also tested the effect of the overexpression of the ADH2 and ADH5 genes on diacid production. Yarrowia lipolytica strains of genotype FT164, poxl-6Δ, dga1Δ, lro1:: URA3, CPR1 were transformed with the alcohol dehydrogenase (ADH) overexpression cassettes pPDX2-ADH2 and pPDX2-ADH5. The strains obtained were verified by PCR and placed in collection.

After characterization of the strains, the inventors tested the diacid production by comparing the strains overexpressing CPR1 and ADH2 and overexpressing CPR1 and ADH5. As a control, the strain overexpressing only CPR1 was used.

The results are presented in FIG. 9.

The results show that the overexpression of ADH2 or ADH5 does not improve DC18:1 production, quite the contrary there is a decrease in diacid productivity, in particular during the overexpression of ADH5. These results are therefore similar to those observed for the overexpression of ALK3 or the overexpression of FALDH3 or FALDH4.

### Example 6—Overexpression of the ALK3+ADH2 and/or ADH5+FALDH3 and/or FALDH4 and/or FAO1 Genes

Despite the negative results obtained, which dissuaded them from using the ALK3 or FALDH3 or 4 genes, the inventors nevertheless tested all the strains which overexpress the entire diacid synthesis pathway.

The strains studied in a first experiment overexpress any one of the following combinations:
ALK3+CPR1+ADH2+FALDH3,
ALK3+CPR1+ADH2+FALDH4,
ALK3+CPR1+ADH5+FALDH3, and
ALK3+CPR1+ADH5+FALDH4.

Flask cultures were carried out and the best results were obtained for the following combinations: ALK3+CPR1+ADH2+FALDH3, ALK3+CPR1+ADH2+FALDH4 and ALK3+CPR1+ADH5+FALDH3. A second production time course was carried out in order to confirm the previous results.

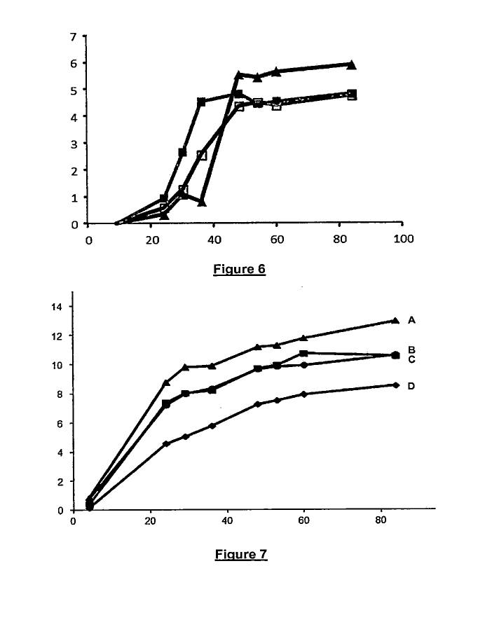

The results obtained for the strains overexpressing ALK3+CPR1+ADH2+FALDH3 or ALK3+CPR1+ADH2+FALDH4 are presented in FIG. 6. The strain used as a control is OLEOX which overexpresses the CPR1 gene.

The diacid production time course showed that the strain overexpressing ALK3+CPR1+ADH2+FALDH3 shows an improvement in the final production of DC18:1 which represents a 20% increase.

The strain overexpressing ALK3+CPR1+ADH2+FALDH4 does not show an improvement in the final production, but it has an increased production rate in the DCA production phase. This represents an advantageous improvement in terms of productivity.

During characterization of the strains obtained, an article was published by Gatter M et al. (Gatter et al., 2014 FEMS Yeast Res. 2014 September; 14(6):858), wherein they identified an enzyme with a fatty alcohol oxidase activity (FAO1). The inventors overexpressed this gene on the pTEF promoter in a strain which overexpresses the CPR1+ALK3 and ADH2 genes.

Three strains were tested for their diacid production capacity, in a flask. A first experiment showed an increase of 25%-60% in the final production of DC18:1 relative to the control strain (OLEOX overexpressing CPR1). The results are represented in FIG. 7.

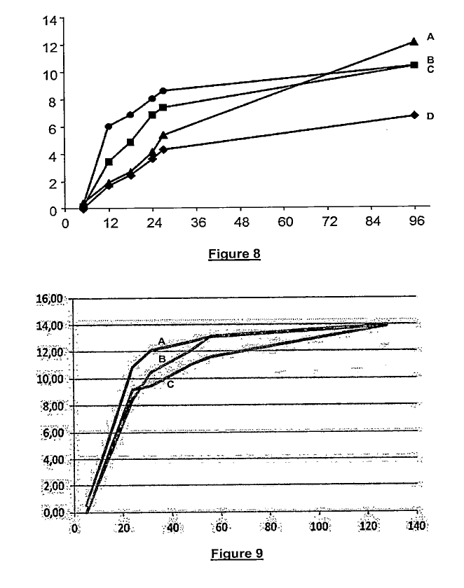

A second experiment allowed the inventors to follow in greater detail the diacid production phase and the previous results were confirmed. Furthermore, a strong improvement in productivity in the 12-24 h of the production phase was observed. The results are presented in FIG. 8.

These results show the production of a strain capable of increasing the production and productivity of DC18:1 and, in addition, that the FAO1 gene plays a very important role for the bioconversion of oleic acid to DC18:1.

The invention is not limited to the embodiments presented and other embodiments will emerge clearly to those skilled in the art.

1-4. (canceled)

5. A method for producing at least one dicarboxylic acid, comprising the following steps:
a) a growth phase, in which is placed in culture a yeast strain incapable of degrading fatty acids, overexpressing at least the following genes:
the ALK3 gene, encoding a cytochrome P450 monooxygenase
at least one of the ADH2 and ADH5 genes, each encoding alcohol dehydrogenases, and
at least one of the FALDH3 or FALDH4 genes, encoding fatty aldehyde dehydrogenases or the FAO1 gene encoding a fatty alcohol oxidase, in a culture medium consisting essentially of an energy substrate which comprises at least one carbon source and one nitrogen source, and
b) a bioconversion phase, in which said yeast strain is brought into contact with at least one fatty acid or a hydrocarbon.

6. The method as claimed in claim 5, further comprising the step of recovering at least one dicarboxylic acid.

7. The method as claimed in claim 5, wherein the fatty acids are in the form of a vegetable oil or of a mixture of alkanes.

8. The method as claimed in claim 5, wherein said yeast is also disrupted for the genes encoding the acyl-CoA oxidase isoenzymes POX1, POX2, POX3, POX4, POX5 and POX6.

9. The method as claimed in claim 5, wherein said at least one fatty acid is a mixture of fatty acids having, by weight, an amount of more than 30% of oleic acid relative to the total weight of the mixture.

10. A composition comprising a mixture of dicarboxylic acids produced by the process of claim 6.

11. A composition comprising:
a first nucleic acid molecule corresponding to the ALK3 gene, encoding a cytochrome P450 monooxygenase
at least one second nucleic acid molecule corresponding to at least one of the ADH2 and ADH5 genes, each encoding alcohol dehydrogenases, and
at least one third nucleic acid molecule corresponding to at least one of the FALDH3 or FALDH4 genes, encoding fatty aldehyde dehydrogenases or corresponding to the FAO1 gene encoding a fatty alcohol oxidase,
said first nucleic acid molecule, second nucleic acid molecule and third nucleic acid molecule being bonded or individualized.

12. The composition as claimed in claim 11, wherein
the first nucleic acid molecule corresponds to the ALK3 gene, said first nucleic acid molecule consisting of the sequence SEQ ID NO: 1 or a molecule having 80% homology with said sequence,
the second nucleic acid molecule corresponds to the ADH2 gene, and essentially comprises or consists of the sequence SEQ ID NO: 5, or to the ADH5 gene, and consists of the sequence SEQ ID NO: 6 or a molecule having 80% homology with said sequence, and
the third nucleic acid molecule comprises the FALDH3 gene, and consists of the sequence SEQ ID NO: 7 or a molecule having 80% homology with said sequence, or to the FALDH4 gene, and consists of the sequence SEQ ID NO: 8 or a molecule having 80% homology with said sequence, or to the FAO1 gene, and consists of the sequence SEQ ID NO: 9 or a molecule having 80% homology with said sequence.

13. A yeast strain transformed by a composition comprising at least one nucleic acid molecule as defined in claim 12.

14. A Yarrowia lipolytica strain chosen from the following strains:
the Y3551 strain, of genotype MATA ura3-302 Ieu2-270 xpr2-322 pox1-6Δ dga1Δ lro1Δ dga2Δ fad2Δ ALK3-LEU2 and of phenotype [Leu+ Ura−],
the Y3950 strain, derived from the Y3551 strain, of genotype MATA ura3-302 Ieu2-270 xpr2-322 pox1-6Δ dga1Δ lro1Δ dga2Δ fad2Δ ALK3-LEU2 CPR1-URA3 and of phenotype [Leu+ Ura+], deposited on Mar. 26, 2015 with the CNCM (Collection Nationale de Culture de microorganismes, [French National Collection of Microorganism Cultures], Institut Pasteur, 25 rue du Docteur Roux, F-75724 PARIS Cedex 15) under number CNCM I-4963,
the Y4428 strain, derived from the Y3950 strain, of genotype MATA ura3-302 Ieu2-270 xpr2-322 pox1-6Δ dga1Δ lro1Δ dga2Δ fad2Δ ALK3 CPR1 and of phenotype [Leu− Ura−],
the Y4457 strain, derived from the Y4428 strain, of genotype MATA ura3-302 Ieu2-270 xpr2-322 pox1-6Δ dga1Δ lro1Δ dga2Δ fad2Δ ALK3 CPR1 ADH2-URA3 and of phenotype [Leu− Ura+], and
the Y4832, Y4833 and Y4834 strains, deposited on March 14, 2016 at the CNCM under the respective numbers CNCM 1-5072, CNCM 1-5073 and CNCM 1-5074, these strains being derived from the Y4457 strain, and having the genotype MATA ura3-302 Ieu2-270 xpr2-322 pox1-6Δ dga1Δ lro1Δ dga2Δ fad2Δ ALK3 CPR1 ADH2-URA3 FAO1-LEU2 and the phenotype [Leu+ Ura+].

15. The method as claimed in claim 5, wherein the fatty acids are in the form of an oil selected from the group consisting of: rapeseed oil, oleic rapeseed oil, sunflower oil, oleic sunflower oil, coconut oil, palm oil, palm kernel oil, olive oil, groundnut oil, soybean oil, corn oil, mustard oil, castor oil, palm olein, palm stearin, safflower oil, sesame oil, linseed oil, hazelnut oil, grapeseed oil, hemp oil, by-products derived from the extraction any of the foregoing oils; and fish oils or oils from yeasts, bacteria or microalgae.

16. The composition as claimed in claim 12, further comprising: a fourth nucleic acid molecule sequence corresponding to the CPR1 gene, consisting of the sequence SEQ ID NO: 14 or a molecule having 80% homology with said sequence.
